(19) United States
(12) Patent Application Publication
(10) Pub. No.: US 2025/0028926 A1
(43) Pub. Date: Jan. 23, 2025

(54) APPARATUS FOR GENERATING A DIGITAL ACCESS ELEMENT

(71) Applicants: BASF SE, Ludwigshafen Am Rhein (DE); BASF COATINGS GMBH, Muenster (DE)

(72) Inventors: Adrian VON MUEHLENEN, Muenster (DE); Victor KAUPE, Muenster (DE); Henning SCHWABE, Ludwigshafen am Rhein (DE); Andreas WOLLNY, Ludwigshafen am Rhein (DE); Dennis HAARDT, Ludwigshafen am Rhein (DE)

(21) Appl. No.: 18/712,831

(22) PCT Filed: Dec. 19, 2022
(86) PCT No.: PCT/EP2022/086721

§ 371 Date: May 23, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06K 19/077

(52) U.S. Cl. CPC G06K 19/0772

(57) ABSTRACT

Disclosed herein are an apparatus for generating a digital access element, a corresponding method, and a computer program.

### FIELD OF THE INVENTION

The invention relates to an apparatus for generating a digital access element, and to a corresponding method and computer program.

### BACKGROUND OF THE INVENTION

In the supply of chemical products, multiple regulatory requirements need to be met which differ depending on the chemical product. For instance, in automotive supply chains, chemical companies provide standardized information using the International Material Data System (IMDS). This system allows to collect data along the entire automotive supply chain. Participants in the automotive supply chain register with the IMDS service and maintain product entries in the central database as provided and hosted by a third-party provider.

Systems like IMDS are, however, static regarding data, prone to error and cumbersome in handling or maintenance. Owing to the highly specific and centralized setup of such systems, exchange and sharing of chemicals data is laborious. Moreover, the centralized setup raises concerns regarding data security. Hence, there is a need to render chemical data exchange and sharing simpler and at the same time more secure. This need is felt particularly by members of supply chains involving coating material production, such as for automobile manufacturing, since these supply chains bridge a technically and logistically wide span from raw materials to the end customer and would therefore particularly benefit from means for a simple and secure data exchange.

### SUMMARY OF THE INVENTION

It is an object of the invention to provide a simpler and more secure way of exchanging data on coating materials.

In a first aspect, an apparatus for generating a digital access element associated with a coating material is presented, wherein the apparatus comprises a) a digital identifier providing unit configured to provide a digital identifier associated with the coating material and coating material data, and b) a digital access element generator configured to generate the digital access element based on the digital identifier and access data.

Since a digital access element for a coating material is generated based on a digital identifier which is associated with the coating material and coating material data, and further based on access data, the digital access element allows for an indirect access to the coating material data, i.e. an access to the coating material data via the digital access element. Access to the digital access element itself can remain unrestricted while still allowing for controlled access to the coating material data. It has been found that such a digital access element allows to exchange coating material data in a simpler and more secure way.

The digital access element could be further defined as a digital data structure allowing third parties to access data on the coating material, wherein the access is usually conditional. The provided digital identifier may be understood as a unique digital reference to a digital identity or a digital twin representing a physical identity of the coating material, or as a unique digital name of the coating material, wherein the digital identity or digital twin representing the physical identity of the coating material may be given in terms of the data on the coating material, i.e. the coating material data. Therefore, and since a passport usually allows to uniquely identify an individual, the digital access element may also be understood as a digital passport for the coating material or as a coating material passport. However, unlike ordinary material passports which list all constituents of a material or even fully characterize the material, the digital access element preferably includes, if at all, only selected data on the coating material itself, but otherwise just access data allowing for access to other coating material data. This can increase data security. For instance, by virtue of digital access elements as described herein, data access can remain under control of data owners. At the same time, controlled data transparency is facilitated by providing means for accessing the data. The digital access element could even be publicly accessible.

Coating materials may include liquid, paste-like or powdery materials which, when applied to at least part of the surface of a substrate, produce a coating with protective, decorative and/or other specific properties (see also DIN EN 971-1:1996-09). Substrates may include metal substrates, plastic substrates and mixtures thereof. The substrates may be pretreated or may comprise at least one coating layer. The coating materials may be applied using commonly known application techniques, such as dipping, bar coating, spraying, rolling or the like. Machines may be stationary machines or movable machines. Stationary machines may include air conditioning devices, power units (nuclear, coal, natural gas, oil, wind, water, solar thermal, geothermal), generators, pumps, hydraulic power units, wind turbines, transformer stations, thermal heat pumps, and compressors. Movable machines may include vehicles. Vehicles may include motor vehicles. Examples of motor vehicles may include motorcycles, cars, trucks, buses, vans, minivans, ATV (all-terrain vehicles) and mobility scooters for disabled people. Vehicles may include railed vehicles. Examples of railed vehicles may include trains and trams. Vehicles may include watercraft vehicles. Examples of watercraft vehicles may include ships, boats and underwater vehicles. Vehicles may include amphibious vehicles. Examples of amphibious vehicles may include screw-propelled vehicles and hovercraft. Vehicles may include aircrafts. Examples of aircrafts may include airplanes, helicopters and aerostats. Vehicles may include spacecraft. The stationary or movable machines may be driven by spark-ignited or self-ignition engines, two- or four stroke engines, electrical engines, fuel cells or combinations thereof (hybrid engines).

In an embodiment, the coating material associated with the digital access element-which could be understood as a coating material passport, as mentioned above, or as a digital asset—may be selected from the group consisting of coating materials and coating material components. Coating materials may be selected from electrocoating materials, primer materials, primer-surfacer materials, filler materials, putty materials, basecoat materials, clearcoat materials or tinted clearcoat materials. For instance, the coating material may be a primer material. In another instance, the coating material may be an electrocoating material. In yet another instance, the coating material may be a clearcoat material. Coating material components may be selected from hardener compositions, additive compositions, thinners, reducers, spot blender compositions, pigment pastes or binder compositions. For instance, the coating material component may be a hardener composition. In another instance, the coating material component may be a binder composition. In yet another instance, the coating material component may be a thinner. In yet another instance, the coating material component may be a reducer. In yet another instance, the coating material component may be a spot blender composition. In yet another instance, the coating material component may be a pigment paste. In yet another instance, the coating material component may be an additive composition.

Electrocoating materials typically comprise an aqueous dispersion or emulsion of film-forming material(s), such as an epoxy resin, having ionic stabilization, such as anionic or cationic stabilization. The film-forming material(s) may be self-crosslinking or may be crosslinkable using a crosslinker. Electrocoating materials may further comprise pigments and/or crosslinking agent(s). The electrocoating materials may be formulated by mixing a binder dispersion comprising the film-forming material(s) and the crosslinker(s) with a pigment paste comprising at least one pigment. During electrodeposition, the electrocoating material is deposited onto a conductive substrate by submerging the substrate in an electrocoating bath containing the electrocoating material and then applying an electrical potential between the substrate and a pole of opposite charge, for example, a stainless-steel electrode. Applying the electrical potential results in the electrocoating composition being deposited onto the conductive substrate. The resulting film may then be cured to obtain a substrate comprising an electrocoating. Suitable electrocoating materials include the electrocoating compositions described in EP0241476B1, EP0675926B1, EP0961797B1, EP1192226B1, WO2021/123106A1 and WO2021/239264A1, each of which is incorporated herein by reference.

Primer materials are typically used to form the first layer of a coating system applied to a substrate or an old coating. The primer materials may be solvent-borne or water-borne. The primer layer typically provides adhesion for the entire coating structure, i.e. not only the primer but also the subsequent layers. In addition, the primer layer may perform different tasks depending on the type of substrate, for example corrosion protection of metal substrates. Primer materials may comprise film-forming material(s), such as polyester, poly(meth)acrylic and epoxy resins. Primer materials may further comprise pigments. Primer materials may further comprise crosslinking agent(s), such as isocyanates and amine components. Primer materials may further comprise at least one adhesion promoter. Suitable primer materials are, for example, described in WO 2019081461 A1, WO 2020234066 A1, WO 2020233939 A1, WO 2021009252 A1, each of which is incorporated herein by reference.

Primer-surfacer materials are coating materials that combine the properties of primers and fillers. The primer-surfacer materials may be solvent-borne or water-borne. The primer-surfacer layer produced from the primer-surface materials may serve to protect against mechanical exposure such as stone chipping, for example, and also to level out unevennesses in the substrate. Primer-surfacer materials may comprise film-forming material(s), such as polyester resins, poly(meth)acrylic resins, epoxy resins and/or poly(meth)acrylic-epoxy resins. Primer-surfacer materials may further comprise pigments. Primer-surfacer materials may further comprise crosslinking agent(s), such as isocyanates and amine components.

Filler materials are typically used to form an intermediate coating layer within a multilayer coating. The filler materials may be solvent-borne or water-borne. The purpose of filler materials is to remove unevenness of the substrate, to support adhesion and corrosion protection (especially if they are used as primer-surfacer materials) as well as to provide good stone chipping resistance of the entire coating system. Filler materials may provide a good and fast sandability. Filler materials may be used wet-on-wet to cover fine sanding marks or smooth filler surfaces. Filler materials may comprise film-forming material(s), such as polyurethane-acrylate(s), polyurethane resins, epoxy resins, polyester resins, polyacrylate resins or a combination thereof. Filler materials may comprise crosslinking agent(s), such as isocyanates, blocked isocyanates or amines. Filler materials may comprise pigment(s). Suitable filler materials are, for example, described in WO 2020234066 A1, WO 2020233939 A1, each of which is incorporated herein by reference

Putty materials may be highly pigmented coating materials used to level out substrate unevenness that is too large and can no longer be corrected by primer materials or filler materials (see also DIN 55945:1996-09). Putty materials may comprise film-forming material(s), such as unsaturated polyester resins or epoxy resins. Putty materials may comprise crosslinking agent(s), such as peroxides or amine components.

Basecoat materials may typically be used as color-imparting intermediate coating materials. The basecoat materials may be solvent-borne or water-borne. The basecoat materials may comprise at least one color pigment and/or at least one effect pigment. The basecoat layer resulting from the basecoat materials may be opaque, i.e. the underlying coating layers may no longer be visible through the basecoat layer. The basecoat materials may comprise film-forming material(s), such as poly(meth)acrylates, polyurethanes, polyesters, poly(meth)acrylate-polyurethanes, polyethers, melamine resins or combinations thereof. Suitable basecoat materials are, for example, disclosed in WO 2018177731 A1, WO 2018197163 A1, WO 2019020324 A1, US20190031910 A1, WO 2019015953 A1, WO 2019207085 A1, WO 2019185306 A1, WO 2019211473 A1, WO 2020151977 A1, WO 2020136016 A1, WO 2020185946 A1, WO 2020216584 A1, WO 2021074187 A1, WO 2021074359 A1, WO 2021078923 A1, WO 2021094131 A1, WO 2021148255 A1, WO 2021191015 A1, WO 2021224232 A1, WO 2022063854 A1, EP 3957693 A1, each of which is incorporated herein by reference.

Clearcoat materials may typically be used as to produce a transparent coating with protective, decorative or specific technical properties (see also DIN EN 971-1:1996-09). Clearcoat materials may not contain color/effect pigment(s) and/or filler(s). Clearcoat materials may contain transparent pigment(s). Clearcoat material(s) may be solvent-based, water-borne or solid materials. The transparent coating compositions preferably comprise at least one (first) polymer as a binder having functional groups, and at least one crosslinker having a functionality complementary to the functional groups of the binder. Preference is given to using at least one hydroxy-functional poly(meth)acrylate polymer as a binder and a polyisocyanate as a crosslinking agent. Suitable clearcoat materials are described, for example, in WO 2006/042585 A1, WO 2009/077182 A1 and WO 2008/074490 A1, each of which is incorporated herein by reference.

Tinted clearcoat materials may typically be used to produce a coating layer which is neither completely transparent and colorless as a clear coating nor completely opaque as a typical pigmented basecoat. A tinted clearcoat layer is therefore transparent and colored or semitransparent and colored. The color may be achieved by adding small amounts of pigments commonly used in basecoat coating materials to clearcoat materials.

Hardener compositions may comprise at least one crosslinking agent. Suitable crosslinking agent(s) may be selected from melamine resin(s), polyisocyanate(s), blocked polyisocyanate(s), polycarbodiimide(s), amine(s) or combinations thereof. The hardener composition may comprise solvent(s), such as organic solvents and/or water.

Binder compositions may comprise at least one binder. The binder may correspond to the nonvolatile component of the dispersion, without pigments and fillers (see also DIN EN ISO 4618:2007-03). The binder may correspond to physically and/or chemically curable polymers, examples being polyurethanes, polyesters, polyethers, polyureas, polyacrylates, polysiloxanes and/or copolymers of the stated polymers. The polymer may comprise anionic, cationic and/or nonionic group(s). The binder may be dissolved or dispersed in a solvent.

Binder dispersions may comprise water as solvent. The binder composition may comprise pigments, such as color and/or effect pigments. The binder composition may comprise a crosslinker, such as a blocked isocyanate. The binder composition may comprise additives commonly used in coating materials, such as the ones described later on. Suitable binder dispersions are described, for example, in EP3197961A1, EP1720923A1, EP3183304A1, WO2021018595A1, WO2021018594A1, US2011042222A1, EP2283087A1, WO2020233939A1, EP3423535A1, WO2020234066A1, WO2020234066A1, WO2016116299A1, WO2022189111A1, WO2015090442A1, each of which is incorporated herein by reference.

Binder solutions may comprise at least one binder dissolved in a solvent. The binder may correspond to the nonvolatile component of the dispersion, without pigments and fillers (see also DIN EN ISO 4618:2007-03). The binder may correspond to physically and/or chemically curable polymers, examples being polyurethanes, polyesters, polyethers, polyureas, polyacrylates, polysiloxanes and/or copolymers of the stated polymers. Suitable binder solutions are described, for example, in WO 2006/042585 A1, WO 2009/077182 A1, WO 2008/074490 A1, WO2021018595A1, WO2021018594A1 and DE4110520A1, each of which is incorporated herein by reference.

Pigment pastes may comprise pigment mixtures in carrier materials, namely different polymers. The concentration of pigments within the pigment paste is typically higher than the concentration of said pigments in a coating material prepared from said pigment paste. More particularly, in such pigment pastes, the weight ratio of pigments to polymers is generally greater than in coating compositions in which the paste is used. The pigment pastes may comprise water and/or organic solvents. The pigment pastes may comprise different additives, such as wetting agents and/or dispersing agents. The pigment pastes may be used to prepare colored coating materials, such as basecoat materials. The pigment pastes may be used within a mixer system to prepare colored coating materials used during the repair process. Suitable pigment pastes are, for example, disclosed in EP3083851A1, U.S. Pat. No. 10,131,808B2, EP3083836A1, WO2018172475A1, US2020140713A1, WO2021018595A1, WO2021018594A1, WO2022175076A1, WO2018019628A1, WO2021123106A1, US2007269606A1, WO2015070930A1 and WO2004018580A1, each of which is incorporated herein by reference.

Additive compositions may comprise at least one additive. The additive may be selected from salts which can be broken down thermally without residue or substantially without residue, reactive diluents, transparent pigments, fillers, dyes soluble in a molecular dispersion, nanoparticles, light stabilizers, antioxidants, deaerating agents, emulsifiers, slip additives, polymerization inhibitors, initiators of free-radical polymerizations, adhesion promoters, flow control agents, film-forming assistants, sag control agents (SCAs), flame retardants, corrosion inhibitors, waxes, siccatives, biocides, and flatting agents, leveling agents, light stabilizers, such as UV absorber(s) or HALS, free-radical scavengers, slip additives, polymerization inhibitors, defoamers, wetting agents, flow control agents, film-forming assistants such as cellulose derivatives, rheology control additives, flame retardants and/or water scavengers, catalysts or a combination thereof. The additive composition may comprise a solvent, such as water or an organic solvent. The additive composition may comprise a binder, such as previously described.

Thinners and reducers are typically used to adjust the viscosity of a coating material to a desired value or range. Thinners may contain solvent(s), such as organic solvents or water. Reducers may contain solvent(s), such as organic solvents or water. The organic solvents may be selected from aliphatic or aromatic hydrocarbon such as Solvesso 100 (trademark), toluene or xylene, an alcohol such as butanol or isopropanol, an ester such as butyl acetate or ethyl acetate, a ketone such as methyl isobutyl ketone or methyl amyl ketone, an ether, an ether-alcohol or an ether-ester, or a mixture of any of these. Suitable reducers and thinners are described, for example, in WO2022200533A1 and WO2011068855A1, each of which is incorporated herein by reference.

Spot blender compositions may typically be applied over the uncured refinish clearcoat to produce “gentle” transition zones and to provide an “invisible” finish leaving no visual indications of repair at the damaged site. The spot blender compositions may comprise film-forming material(s), such as polyols, urethane (meth)acrylates or mixtures thereof. The spot blender compositions may comprise crosslinking agent(s), such as polyisocyanate(s), and may not comprise film-forming material(s), e.g. may be free of film-forming material(s). The spot blender composition may comprise a solvent, such as an organic solvent. The organic solvent may be selected from n-butyl acetate, isobutyl acetate, and xylene. Suitable spot blender compositions are described, for example, in US2010216945A1, WO2006097386A1, WO2006097385A1 and WO2007027286A2, each of which is incorporated herein by reference.

In an example, the digital identifier is a decentralized identifier (DID) as described, for instance, in the W3C recommendation dated 19 Jul. 2022. The digital access element may then be regarded as a DID document. The subject of the digital identifier, i.e. in that case of the DID, however, is then not any generic subject, but specifically a coating material. In another example, the digital identifier is a Universally Unique Identifier (UUID) such as, for instance, according to the UUIDv4 standard. The digital identifier may be associated with one or more decentralized identifier(s) (DID(s)), for instance with one or more DID(s) of data sets contained in the digital twin of the coating material. The digital identifier may be associated with one or more Universally Unique Identifier(s) (UUID(s)), for instance with one or more UUID(s) of data sets contained in the digital twin of the coating material. The digital identifier may be associated with one or more decentralized identifier(s) (DID(s)) and one or more Universally Unique Identifier(s) (UUID(s)), for instance with one or more DID(s) of data sets and one or more UUID(s) of data sets contained in the digital access element of the coating material. Data exchange may include discovery of the digital identifier(s) by participant nodes of the decentral network, authentication of participant nodes of the decentral network and/or authorization of data transfers via a peer-to-peer communication between participant nodes of the decentral network.

The digital identifier is associated with the coating material, and particularly with its digital and/or physical identity. Moreover, the digital identifier is associated with data on the coating material, i.e. coating material data. Besides, the digital identifier may also be associated with an owner (e.g. data owner) of the coating material data. The digital identifier may be associated the coating material producer. The digital identifier may be associated with a machine, a system, or a device used for producing the coating material, or a collection of such machine(s), device(s) and/or system(s). In fact, the digital identifier may be a unique identifier uniquely associated with the coating material data and the owner of the coating material data. Via the digital identifier and its preferably unique association with the data owner and the coating material data, access to the coating material data may be controlled by the data owner. This contrasts with central authority schemes, where identifiers are provided by a central authority and access to data is controlled by such central authority. While digital identifiers based on which digital access elements shall be generated as described herein may also be issued by a central identity issuer, access to the coating material data shall at least not be controlled by this central identity issue. It may be preferred that the digital identifier based on which a digital access element is generated is issued by a decentral identity issuer. The term “decentral” refers in this context to the usage of the identifier in implementations as controlled by the data owner.

A data owner may be any entity generating data, particularly coating material data. Hence, by generating the data, an entity may become the owner of the generated data. The data, particularly the coating material data, may be generated by a third-party entity on behalf of the entity owning physical products from or for which data is generated. Generating data may refer to data acquisition, such as by measurements, but could also refer to a processing of data if the processing leads to new data, wherein “new data” may be characterized by new technical information as compared to the non-processed data. The data generating node may be coupled to the entity owning coating materials from or for which data, particularly, the coating material data, is generated. Instead of being derived from who generated the data, data ownership may also derive from ownership over a production producing the coating material. Hence, for instance, if the producer of the coating material does not acquire or otherwise generate the coating material data, but if instead a third-party entity acquires or otherwise generates the coating material data, the producer of the coating material may be the owner of the coating material data. The coating material data may be accessible for the data owner. The data owner may hence directly or indirectly own the coating material data. The coating material data may be stored in a data base of or associated with the data owner. The coating material data may be stored in a data base accessible by the data owner. Via the digital identifier and its unique association with the data owner and coating material data, access to the coating material data may be controlled by the data owner. For instance, the data owner may control access to the coating material data, for example via a data providing service of the data owner. The coating material data may be associated with the data owner. The data owner may be the owner of the coating material data or the coating material data owner. In this sense, the data owner is to be construed broadly as the entity having access to the coating material data and controlling access by data consuming services of the decentral network to the coating material data. The apparatus for generating the digital access element associated with a given coating material may be coupled to an entity producing the coating material. The apparatus for generating the digital access element associated with a given coating material may be coupled to an entity generating the digital access element on behalf of the data owner, such as the coating material producer.

The access data may refer to any data for accessing the coating material data. The access data may be indispensable, i.e. strictly necessary for accessing the coating material data. Alternatively, the access data may be suitable for accessing the coating material data, while the coating material data could also be accessed in different ways, without the access data based on which the digital access element is generated. Access data may include an endpoint for data exchange or sharing (resource endpoint) or an endpoint for service interaction (service endpoint), that is uniquely identified via a communication protocol. Access data may include authorization schemes and/or cryptographic information. The access data may be provided to the data consuming service. The access data may be provided by a decentral network database, a database associated with the data consuming service, the data providing service associated with the data owner or combinations thereof. Exemplary access data include a digital address or storage location of the coating material data, data relating to authentication and authorization schemes and cryptographic information. For instance, the access data may include a public key, such as a public key needed for decrypting the coating material data. The digital identifier and the access data may be associated with each other. Hence, for instance, the digital identifier based on which a digital access element is generated may be associated with authentication information which is used as access data based on which the digital access element is generated.

It shall be understood that the apparatus for generating the digital access element associated with the coating material may comprise one or more computing nodes and one or more computer-readable media having thereon computer-executable instructions which, when executed by the one or more computing nodes, cause any unit of the apparatus, particularly the digital identifier providing unit and the digital access element generator, to carry out the respective functions for which they are configured. The apparatus for generating the digital access element associated with a given coating material may be coupled to an entity producing the coating material. The apparatus for generating the digital access element associated with a given coating material may be coupled to an entity generating the digital access element on behalf of the data owner, such as the coating material producer.

The apparatus for generating the digital access element associated with the coating material may further comprise, i.e. apart from the digital identifier providing unit and the digital access element generator, a physical identifier providing unit configured to provide a physical identifier for the coating material, wherein the digital identifier is provided based on the physical identifier. For instance, an identifier element may be physically attached to a container of the coating material, wherein the identifier element may contain or correspond to the physical identifier. The physical identifier providing unit may be configured to provide the physical identifier based on the identifier element, such as by use of an ID reader, for instance.

The digital identifier may be associated with the coating material via the physical identifier. The physical identifier may refer, for instance, to a batch number of the coating material. The identifier element may comprise a passive or active element such as, for instance, a barcode, a QR code, an embossed code, an optical holographic code, such as zero-order diffractive microstructures, or an RFID tag. A digital identifier provided based on a physical identifier is, however, not limited to being equal to or comprise the physical identifier. Optionally, the digital identifier is assigned to the physical identifier. In order to assign a digital identifier to a physical identifier, the apparatus for generating the digital access element associated with a respective coating material may comprise an assigning unit, i.e. an assignor. This allows to associate the digital identifier to the physical identifier associated with the coating material. The assigning unit may be contained within the physical identifier providing unit or may be separate from said physical identifier providing unit. The assigning unit, or assignor, may be configured to assign a physical identifier to a digital identifier. Hence, an assignment between the physical identifier and the physical identifier may be carried out by the assignor in any or both of the two directions. Assigning may include generating a code having embedded the digital identifier received from the digital identifier providing unit. Assigning may include correlating the digital identifier received from the digital identifier providing unit with the physical identifier received from the physical identifier providing unit. For instance, an identifier element may be physically attached to a packaging of the coating material, wherein the identifier element may contain or correspond to the physical identifier. The identifier element may comprise a passive or active element such as, for instance, a barcode, a QR code, an embossed code, an optical holographic code, such as zero-order diffractive microstructures, or an RFID tag. The physical identifier providing unit may be configured to provide the physical identifier based on the identifier element, such as by use of an ID reader. The determination, or acquisition, of the physical identifier may be viewed as triggering the providing of the digital identifier, and possibly also a subsequent assignment between the physical identifier and the digital identifier.

The digital identifier may be associated with the physical entity the coating material will be supplied for and the coating material data is associated with. For instance, the digital identifier may be associated with the physical entity of the component, the component assembly, the end product or the like produced using the coating material. The digital identifier may be associated with more than one physical entity the coating material will be supplied for and the coating material data is associated with. For instance, the digital identifier may be associated with the physical entity of the component, the component assembly and the end product produced using the coating material. Associating the digital identifier with different physical entity stages in the chemical supply chain allows for virtually tracking the supplied coating material in the supply chain. This way coating material with its associated coating material data may be tracked e.g. up to the end of life of the end product.

The apparatus may also comprise a collector configured to collect coating material data, wherein the digital identifier is associated with the collected coating material data. In an example, the collector may comprise manual, semiautomatic or automatic input means for inputting the coating material data. The input means may comprise one or more sensors, particularly measurement devices such as, for instance, for measuring a state of matter of a coating material, its density, its colour, et cetera. Optionally, the collector is configured to collect the coating material data based on a physical identifier or a coating material identifier. If the physical identifier corresponds to an identifier element physically attached to a container of a coating material, for instance, the collector may comprise an ID reader for reading out the identifier element. More generally, the collector may comprise, or cooperate with, the physical identifier providing unit. For instance, the physical identifier providing unit may be configured to provide a physical identifier corresponding to an identifier element physically attached to packaging of the coating material, wherein the collector may be configured to collect the coating material data based on the provided physical identifier. Optionally, the coating material data, or a part thereof, could be already encoded in the physical identifier, particularly in the identifier element physically attached to a packaging of the respective coating material, wherein the collector may be configured to collect the coating material data, or the part thereof, by decoding it from the physical identifier.

The digital identifier providing unit may be configured to provide the digital identifier upon receiving a request to provide at least said digital identifier. Hence, the digital identifier providing unit may provide, apart from the digital identifier, further data. The further data may be used by the digital access element generator to generate the digital access element. The further data may include access data as described above and/or later on. The request may be received by the digital identifier providing unit and/or the digital access element generator via any communication channel, including but not limited to web servers, ad hoc WIFI, BLE beacon signals, NFC, a barcode, QR code RFID code scanning, etc. The request to provide the digital identifier may be associated with the coating material production producing the coating material. The request to provide the digital identifier may be generated by a computing system, such as an operating system, of the coating material production producing the coating material. The request may be triggered at predefined occurrences. For instance, the packaging/filling line may comprise a detector detecting the physical identifier on each packaging unit of the coating material. Based on such recognition a computing apparatus, such as the operating system, of the coating material production may generate the request to provide the digital identifier. The generated request may be provided to the digital identifier providing unit.

The request to provide the digital identifier may include access data and/or an owner or product identifier associated with the coating material data owner or the coating material, respectively. The owner/product identifier may be a string identifier associated with a data owner name, such as the coating material producer. The owner identifier, like the digital identifier associated with the coating material, may be provided based on a physical identifier, such as based on a barcode, a QR code, an embossed code, an optical holographic code, such as zero-order diffractive microstructures, or a tag like an RFID tag. Through the owner identifier the digital access element generated can be associated with the coating material data owner by including the owner identifier. The owner/product identifier may be used for data transactions, such as sharing or exchanging coating material data. The owner/product identifier may be provided to a transaction manager. Providing the digital identifier and the owner/product identifier of the data owner to a transaction manager or a data consuming service may simplify tracking of data transactions. Any transaction in the data ecosystem can e.g. be associated with the clear name of the data owner or the coating material.

The request to provide the digital identifier may further contain data associated with the detected physical identifier, such as the coating material ID or batch number. The request to provide the digital identifier may further contain an indication of the type of digital identifier, such as DID or UUID.

The request may contain an authentication mechanism, such as the public-private key pair, and/or an identifier associated with the digital identifier providing unit and/or the digital access element generator. This allows to associate the request to a specific digital identifier providing unit and/or a specific digital access element generator. The authentication mechanisms may be retrieved from an authentication data storage, such as a vault, depending on the digital identifier to be requested.

In response to the request, the digital identifier providing unit may generate the digital identifier. Hence, the digital identifier providing unit may include a digital identifier generating unit. The digital identifier may be generated at a central node or one or more decentral nodes. The one central node or the decentral node(s) may be part of a decentral system comprising a plurality of member nodes. The digital identifier generating node may be part of the apparatus described herein or may be separate from said apparatus. The digital identifier may further be provided to at least one authentication data registry node, preferably accessible by the data providing service and/or the data consuming service. The authentication data registry node may be a central registry node such as a central file system, a centrally managed distributed database, and/or a centrally managed peer-to-peer network. This enables customized data sharing or exchange with respect to the coating material and the chemical supply chain the coating material is supplied to. In particular, the data providing service and/or the data consuming service may customize data sharing or exchange protocols based on the anchoring of the digital identifier to the coating material data. The authentication data registry node may be a decentral registry such as a distributed ledger, a decentralized file system, a distributed database, and/or a peer-to-peer network. The decentral configuration allows for more efficient use of computing resources and strengthens control by the data owner. In addition, the decentral configuration is independent from centrally managed nodes and as such increases reliability and flexibility of the system. The authentication data registry node may be a central registry node such as a central file system, a centrally managed distributed database, and/or a centrally managed peer-to-peer network. The central configuration allows for more control and standardization via a central node.

The digital access element may include the digital identifier and the access data. Hence, the digital access element generator may not only be configured to generate the digital access element based on the digital identifier provided by the digital identifier providing unit and access data, but also to generate the digital access element such that it includes the digital identifier and the access data. In this way, the generated digital access element associated with the respective coating material will include a) the digital identifier associated with the coating material and coating material data, and b) access data. This allows for a direct and therefore relatively simple transmittal of the digital identifier and the access data to a data consumer or any other party for which the digital access element may be generated. The generated digital access element may contain further data, such as an owner or product identifier. This allows to associate any transaction in the data ecosystem with the clear name of the data owner or the coating material. The generated access element may contain authentication mechanism(s) associated with the digital identifier.

The access data may allow for an access to at least part of the coating material data. Other parts of the coating material data may already be included in the digital access element. Such data, i.e. the data already included in the digital access element, may, however, only be accessible using the access data based on which the digital access element is generated. Hence, both, the part a) of the coating material data accessible via the digital access element without being included in the digital access element and the part b) of the coating material data already included in the digital access element, may only be accessible using the access data. Different parts of the access data may be necessary for each of the two aforementioned parts of the coating material data. Other, i.e. yet further, parts of the coating material data may not be accessible at all due to, for instance, not being acquired, collected, stored and/or digitalised. In one embodiment, the access data includes one or more digital representation(s) pointing to coating material data or parts thereof. In this context pointing means any network representation or address that is suitable for accessing the coating material data. The access data may include multiple digital representations pointing to distinct parts of the coating material data. The access data may include multiple digital representations pointing to different parts of the coating material data. Such different parts may overlap in some data points. The representation may include an access point to the coating material data, a link to access coating material data, an endpoint to access coating material data or a service endpoint to access coating material data. This way the coating material data can be maintained and controlled by a data owner, such as the coating material data owner. Access can be provided via the representation of an access point simplifying data verification, integrity checks or quality checks and access control, since not multiple distributed data points need to be checked and access controlled. The coating material data may be stored in a data base of or associated with the data owner. The coating material data may be stored in a data base accessible by the data owner. The digital representation pointing to coating material data or parts thereof may be associated with or relate to any such data base associated with or accessible by the data owner. For enhanced security the digital representation pointing to coating material data or parts thereof may indirectly relate to any such data base associated with or accessible by the data owner. Access data may include data relating to authentication and authorization schemes and/or cryptographic information such as, for instance, a public key for decrypting the coating material data. For instance, the access data may include any data used in authentication methods according to W3C recommendations, and/or any data needed for accessing data providing services like the ones known from IDS architectures, particularly their addresses.

The digital access element may be related to or includes one or more authentication mechanisms. The authentication mechanisms may be associated with the digital identifier and/or the access data. The authentication mechanism may directly or indirectly relate to the digital identifier and the access data. In an example of indirect relation, the authentication mechanism may relate to a certificate mechanism. For example, on access request by the data consuming service, a dynamic access token may be generated based on the certificate mechanism. Such dynamic access token may be used to open peer-to-peer communication channel between data consuming service and the data providing service. The authentication mechanism may include a token, such as private and public key infrastructure, a certificate mechanism or a biometric mechanism, such as fingerprints, face recognition or voice recognition or the like. One common public key certificate is for instance the X.509 certificate. Through the authentication mechanism data access by a data consuming service can be controlled in a secure manner and integrity of the data providing service can be ensured. This allows for more reliable, controlled and secure data exchange or sharing.

The one or more authentication mechanisms associated with the digital identifier may be provided to the digital access element generator and to at least one decentral authentication data registry, preferably accessible by the data providing service and/or the data consuming service. The authentication data registry may be a central registry such as a central file system, a centrally managed distributed database, and/or a centrally managed peer-to-peer network. The central configuration allows for higher control and standardization via a central node. The authentication data registry may be a decentral registry such as a distributed ledger, a decentralized file system, a distributed database, and/or a peer-to-peer network. The decentral configuration allows for more efficient use of computing resources and strengthens control by the data owner.

The digital access element may be related to or includes one or more authorization mechanisms. The authorization mechanism(s) may be associated with the decentral identifier and/or the access data. The authorization mechanisms may include authorization rule(s) including data transaction instructions or data transaction protocols, such as data usage policies, smart data contracts or more complex data processing instructions associated with data providing and/or data consuming services. Through the authorization mechanism data access and data usage by a data consuming service can be controlled in a secure manner. The one or more authorization mechanisms associated with the digital identifier may be provided to a node for generating or processing the digital access element or for accessing the coating material data. Additionally or alternatively, the one or more authorization mechanisms may be provided to at least one central or decentral authorization data registry, preferably accessible by the data providing service and/or the data consuming service. The one or more authorization mechanisms may be provided to a node generating or processing the digital access element and to at least one of a central file system, a centrally managed distributed database, a centrally managed peer-to-peer network, a distributed ledger, a decentralized file system, a distributed database, and/or a peer-to-peer network, preferably accessible by the data providing service and/or the data consuming service.

The digital identifier providing unit may be configured to provide-apart from the digital identifier associated with the coating material and the coating material data (hereinafter referred to as first digital identifier)—at least one precursor digital identifier associated with one or more precursor materials from which the coating material is produced. The digital identifier providing unit may be configured to generate a concatenation of the first digital identifier and precursor identifier(s) based on a relationship representation according to which the first digital identifier is associated with the precursor identifier(s). The relationship representation may be associated with a relationship between the coating material and each precursor material. The relationship representation may specify that the precursor material(s) may be used to produce the coating material and/or that the coating material may be produced by using the precursor material(s). One or more hash value(s) may be generated based on the relationship representation. Hash value(s) may be generated based on the concatenation of digital identifiers. Hash value(s) may be generated for the concatenation of digital identifiers. Hash value(s) may signify the concatenation of digital identifiers. The hash values may be generated based on data associated with the first digital identifier and the precursor material digital identifier(s). The hash value may be generated based on a combined data set associated with the first digital identifier and the precursor material digital identifiers. By concatenating digital identifiers associated with products of the chemical product supply chain and/or the recycling product chain, the physical entities can be tracked and traced virtually.

The digital identifier providing unit is configured to provide the first digital identifier and the at least one precursor material identifier to the digital access element generator. The digital identifier providing unit may be configured to provide the first digital identifier and the at least one precursor material digital identifier to a retrieval data storage (also denoted as authentication registry within this disclosure). The request to provide the digital identifier may include the one or more precursor digital identifiers. The request to provide the digital identifier may include data associated with the physical identifier of the coating material. Based on said data associated with the physical identifier, the digital identifier providing unit may retrieve the one or more precursor digital identifiers.

A digital identifier associated with a precursor material may be associated with precursor material data indicative of whether the precursor material is a virgin or a recycled material, of an environmental impact and/or of an origin of the precursor material. The digital identifiers associated with the precursor materials may be determined based on physical identifiers possibly corresponding to physical identifying elements like tags attached to the packaging of the precursor materials, for instance. Hence, the digital identifier providing unit may be configured to determine the digital identifiers associated with the respective precursor materials.

The precursor materials may be raw materials, including virgin or recycled materials. Raw materials may include pigment(s), monomers used to prepare polymer(s), solvents and coating material additives. The precursor materials may be intermediate products manufactured from raw material(s) and/or other intermediate product(s). Intermediate products may include polymer solutions and/or polymer dispersions. The polymers may be selected from film-forming polymers, such as alkyd resins, polyester resins, polyimides, silicone resins, novolak resins, urea resins, melamine resins, amino resins, polyurethane resins, epoxy resins, polyolefin resins, polyvinyl resins, polyacrylic resins, polymethacrylic resins, or copolymers thereof. Intermediate products may include pigment pastes produced from polymer solutions and/or polymer dispersions and pigment(s). Intermediate products may include base varnishes produced from polymer solutions and/or polymer dispersions. The coating material may be produced from raw materials and intermediate products. For instance, the coating material may be produced from pigment paste(s) and a base varnish. In another instance, the coating material may be produced from polymer solutions and/or dispersions and raw materials, like solvents and coating material additives. Like the coating material for which the digital access element is generated, also the precursor materials preferably have digital identifiers associated to them, which are referred to as precursor digital identifiers. A digital identifier associated with a precursor material may be associated with precursor material data indicative of whether the precursor material is a virgin or a recycled material, of an environmental impact and/or of an origin of the precursor material.

Also, the digital identifiers associated with the precursor materials may be determined based on physical identifiers possibly corresponding to physical identifying elements like tags attached to the packaging of the precursor materials, for instance. The “manufacturing” of the coating material from the precursor material could also be referred to as a production, i.e. a chemical production of the coating material in which the precursor materials are used to produce the coating material.

The precursor materials may be used directly or indirectly for manufacturing the coating material. That is to say, some of the precursor materials may be used for manufacturing one or more further precursor materials based on which, in turn, the coating material for which the digital identifier is to be provided is manufactured as described previously. Hence, at least part of the precursor materials used to produce the coating material may be intermediate products manufactured from raw material(s) and/or other intermediate product(s).

The digital access element generator may be configured to generate the digital access element based on the first digital identifier (e.g. the digital identifier associated with the coating material and the coating material data) and the at least one precursor identifier or the generated concatenation. This allows to link the digital identifier associated with the coating material with precursor digital identifiers associated with precursor materials used to produce the coating material. Hence, the relationship between the coating material and precursor materials used to produce the coating material can be reflected within the digital access element.

The coating material data may include data on any of: the one or more precursor materials, the coating material itself, a transport of the one or more precursor materials to a manufacturing site of the coating material, a further transport of the manufactured coating material to a consumption location (e.g. a location for manufacturing further products, such as coated substrates, using the received coating material). The expression “any of”, when followed by a list of elements, is to be understood herein to include any one of the listed elements individually as well as any combination of the listed elements.

The data on the one or more precursor materials may include data indicative of any of the following:
a shipper for the transport of the precursor material to a manufacturing site of the coating material for which the digital access element is generated,
a consignee for the transport of the precursor material to a manufacturing site of the coating material for which the digital access element is generated,
components of the precursor material, particularly corresponding charges/batches, such as by means of charge/batch names of the precursor material and/or its components, charge/batch numbers of the precursor material and/or its components, a chemical composition of the precursor material and/or its components, and/or certifying documents/information,
one or more packaging materials and/or an amount of handling units used for the transport of the precursor material to the manufacturing site of the coating material for which the digital access element is generated, wherein exemplary handling units include pallets, drums and intermediate bulk containers (IBCs),
a weight (e.g., brutto and/or netto) of the precursor material, particularly per handling unit,
a value of the precursor material and/or one or more of its components,
a risk associated with the precursor material, i.e. a dangerous goods information,
a condition of the precursor material, such as a raw material condition as determined in a visual logistics check,
one or more precursor material constituents, such as a raw material composition as determined in an analytical quality management (QM) check carried out in a lab,
emission data of the precursor material,
recycled content data of the precursor material,
production data of the precursor material.

Emission data may comprise any data related to an environmental footprint, such as the carbon footprint, of the coating material. Emission data may include data relating to greenhouse gas emissions e.g. released in production of the chemical product. Emission data may include data related to greenhouse gas emissions. Greenhouse gas emissions may include emissions such as carbon dioxide (CO₂) emission, methane (CH₄) emission, nitrous oxide (N₂O) emission, hydrofluorocarbons (HFCs) emission, perfluorocarbons (PFCs) emission, sulphurhexafluoride (SF₆) emission, nitrogen trifluoride (NF₃) emission, combinations thereof and additional emissions. Emission data may include data related to greenhouse gas emissions of an entity's or company's own operations (production, power plants and waste incineration). This defines a first scope, i.e. scope 1. Scope 2 comprise emissions from energy production which is sourced externally. Scope 3 comprise all other emissions along the value chain. Specifically, this includes the greenhouse gas emissions of raw materials obtained from suppliers. Product Carbon Footprints (PCFs) sum up greenhouse gas emissions and removals from the consecutive and interlinked process steps related to a particular product. Cradle-to-gate PCFs sum up greenhouse gas emissions based on selected process steps: from the extraction of resources up to the factory gate where the product leaves the company. Such PCFs are called partial PCFs. In order to achieve such summation, each company providing any products must be able to provide the scope 1 and scope 2 contributions to the PCF for each of its products as accurately as possible, and obtain reliable and consistent data for the PCFs of purchased energy (scope 2) and their raw materials (scope 3).

Production of precursor materials may include recycling processes. Recycling processes may include re-use processes and recycling processes. Recycling processes may include a physical and/or chemical treatment of the material to be recycled. Production data may include monitoring and/or control data associated with the production of the precursor material. Production data may include measurement data related to a quality of the precursor material. Production data may include time and location data associated with the production. Recycled content data may comprise any data related to the recyclate content used for providing or producing the coating material. For instance, the recyclate content of a coating material may stem from using recycled precursor materials or precursor materials containing a recycled content.

The data on the coating material for which the digital access element is generated may include data indicative of any of the following:
a shipper for the further transport of the coating material,
a consignee for the further transport of the coating material,
a producer of the coating material, such as a name and/or an address and/or ID of the producer, for instance,
a filling date, i.e. a date on which a coating material has been filled into a packaging in which it is transported and/or sold,
a best-before and/or a use-by date for the coating material,
a name, number, charge, etc., of the coating material, and/or certifying documents/information relating to the coating material,
a chemical composition of the coating material, such as name, number, charge, etc., of it its components, and/or certifying documents/information relating to the chemical composition of the coating material and/or its components,
one or more packaging materials and/or an amount of handling units used for the further transport of the coating material, wherein exemplary handling units include pallets, drums and intermediate bulk containers (IBCs),
a weight (e.g., brutto and/or netto) of the coating material, particularly per handling unit,
a value of the coating material and/or one or more of its components,
a risk associated with the coating material, i.e. a dangerous goods information, which may, particularly for Germany, include a water pollution class,
certificate of analysis data, such as determined in an analytical quality management (QM) check carried out in a lab,
production data for the coating material, such as, for instance, manufacturing parameters for the manufacturing of the coating material,
material safety data sheet for the coating material,
a registration status of the coating material, such as in accordance with legal regulations like REACH in Europe,
information that is necessary for customs clearance of the coating material, such as, for instance, certificates and recipes for the coating material,
one or more properties of the coating material, wherein the one or more properties may refer, for instance, to chemical and/or physical properties such as a boiling point, a solid content, a solvent content, a glass transition temperature, an isocyanate (NCO) content, a storage stability, suitable storage conditions, etc.,
information which is relevant for a further use of the coating material such as, for instance, suitable curing temperatures, a dilution ratio, the material's solubility, etc.,
emission data of the coating material.

The data on the transport of the one or more precursor materials to a manufacturing site of the physical entity for which the digital element is generated, and/or on the further transport of the coating material for which the digital access element is generated to a consumption location, may include data indicative of any of the following:
a shipper for the respective transport,
a consignee for the respective transport,
an amount of handling units used for the respective transport, wherein exemplary handling units include pallets, drums and intermediate bulk containers (IBCs),
a risk associated with the respective transport, which may include dangerous goods information,
a date and time of at least one of arrival, loading and shipping,
information about any deficiencies of the respectively transported material,
a shipment number of the respective transport,
loading information for the respective transport, such as, for instance, one or more loading pictures, information on a loading safety, driver information, a driver quality assessment,
a mode of the respective transport, particularly whether the respective transport is carried out via the sea, the air or via land,
a company name of the respective one or more carriers,
a truck style and equipment used for the respective transport, such as, for instance, whether the truck or equipment is suitable for any dangerous goods transported and/or whether the truck is able to maintain a necessary temperature/transported physical entity,
whether the respective transport is a direct transport or a hub transport, and/or via which ports, airports and/or hubs the respective transport is carried out,
information regarding a main run or main leg of the respective transport, such as, for instance, a ship or plane number used for the main run or main leg,
an estimated time of arrival for the respective transport,
a temperature monitoring, such as a temperature monitored with a sensor device attached to the respective handling unit, wherein, according to one option, the complete temperature monitoring data is provided and/or included, and according to another option, only an information of whether the temperature monitoring was acceptable or not is provided and/or included,
a route monitoring, such as position data monitored with a sensor device attached to the handling unit, wherein, according to one option, the complete position data is provided and/or included, and according to another option, only an information of whether the monitored position was acceptable or not is provided and/or included,
a shock monitoring, such as shock or acceleration data monitored with a sensor device attached to the handling unit, wherein, according to one option, the complete shock or acceleration data is provided and/or included, and according to another option, only an information of whether the monitored acceleration was acceptable or not—such as whether there have been any shocks or not—is provided and/or included,
a monitoring of whether a respectively transported material, particularly its packaging, was opened or damaged,
a CO2 emission during the respective transport,
participating logistics partners for the respective transport, such as third party logistics (3PL) partners, fourth party logistics (4PL) partners and/or carriers, for instance,
a bill of lading, particular its content, for the respective transport,
a proof of delivery for the respective transport.

The data on the one or more precursor materials, data on the coating material itself and/or data on the transport of the precursor materials and/or the coating material may include different classes of said data. For instance, at least one class of data on the coating material itself may include data required by regulation or regulatory data for chemicals, such as declaration data, safety data and certificate of analysis data. At least one further class of data on the coating material itself may include emission data, recyclate content data, bio-based content data, production data and/or composition data of the coating material and/or precursor material(s) used to produce the coating material. Access to the data on the coating material itself may be controlled based on said classes. For instance, access to emission data, recyclate content data, bio-based content data, production data, composition data of the coating material and/or precursor material(s) used to produce the coating material or combinations thereof may be restricted while access to data required by regulation or regulatory data may not be restricted. Such access restriction may be provided by an authorization mechanism or scheme. For instance, the authorization mechanism or scheme may include a rule that specifies which data consuming services get access under which conditions.

In another instance, at least one class of data for the transport of the precursor and/or the coating material may include an indication of whether one or more certain condition(s), such as temperature, shock or transport route, has/have been monitored during transport. At least one further class of data for the transport of the precursor and/or the coating material may include monitoring data, such as position data, shock data and/or route data acquired with a sensor device attached to the handling unit. Access to the data for the transport of the precursor and/or the coating material may be controlled based on said classes as previously described. For instance, access to the monitoring data may be restricted while access to data indicating whether certain condition(s) has/have been monitored may not be access restricted.

Access to the coating material data via the digital access element may be controlled by a data providing service. The data providing service may comprise computer-executable instructions for providing and/or processing data, such as coating material data, associated with the data owner for accessing and/or processing by a data consuming service. The data providing service may be associated with the data owner of the coating material data, such as the coating material producer. This allows to retain full control over the coating material data by the data owner but at the same time enabling sharing of the coating material data under controlled conditions, for example by using appropriate authorization and authentication mechanisms or schemes, to improve production processes, such as discrete manufacturing, or recycling processes of discrete products containing the coating material, either in reacted or in unreacted form, associated with the coating material data.

The digital access element may be provided for access by a data consuming service. The data consuming service may comprise computer-executable instructions for accessing and/or processing data, such as coating material data, associated with a data owner, such as the coating material data owner. The data consuming service may be owned or controlled by a consumer of the coating material, such as a discrete product producer or a participant of the recycling process of the discrete product. A discrete product may be broken down at the end of its lifecycle so that its components can be recycled. Examples of discrete products include automobiles, airplanes, shoes, etc. The data consuming service may comprise computer-executable instructions for accessing and/or processing data, such as coating material data received from the data providing service. The data consuming service may be controlled or owned by a discrete product processor. The coating material data received by the data consuming service may be stored on a dedicated storage owned or controlled by the entity owning or controlling the data consuming service. Via the data consuming service, the discrete product processor is able to retrieve the coating material data associated with received coating material(s). Use of the coating material data associated with the digital access element(s) during production of discrete materials or components thereof allows to improve production processes using said data, for example by controlling the production processes based on said coating material data, to enhance the properties of the resulting coated component or discrete product or the overall production efficiency. Use of the coating material data associated with the digital access element(s) during recycling processes allows to reliably determine the chemical composition of the components to be recycled, thus improving recycling efficiency by determining the correct recycling process, recycling parameters, recycling plant, etc.

The apparatus for generating the digital access element may be comprised in an apparatus for manufacturing the coating material, i.e. in a manufacturing apparatus. The manufacturing apparatus may refer to one or more reactors or other equipment in a production facility producing the coating material. During manufacturing of the coating material, or in the course of its subsequent packaging, a physical identifier may be attached to it, either manually, semi-automatically or automatically. The digital identifier providing unit may be configured to provide the digital identifier for the manufactured coating material based on this physical identifier. Moreover, coating material data may be collected during and/or after manufacturing of the coating material, such as using a collector as indicated above. The provided digital identifier may hence be associated with this coating material data. Thus, apart from the coating material being physically manufactured, a digital access element is generated for the coating material being manufactured.

The apparatus for generating the digital access element, and thereby also the apparatus for manufacturing the coating material and comprising the apparatus for generating the digital access element, may cooperate with an apparatus for managing digital access elements for coating materials, particularly for managing those digital access elements which have been generated by the apparatus for generating digital access elements. Instead of cooperating with a further apparatus, a single combined apparatus may be provided, wherein the single combined apparatus may fulfil the functions of generating and managing the digital access elements. The apparatus for managing digital access elements, or the single combined apparatus, may comprise a digital access elements storage for storing the digital access elements, and/or a retrieval data storage for storing retrieval data associated with the digital access elements. The retrieval data may allow for a retrieval of the digital access elements from the digital access elements storage. The retrieval data may allow for retrieval of the coating material data from a data providing service using the stored retrieval data by a data consuming service. By storing the digital access elements and allowing for a retrieval of the stored digital access elements, a necessity to repeatedly generate the digital access elements may be avoided, which can simplify exchanging and sharing of coating material data.

It may also be preferable to not store the digital access elements. This may increase data safety. Then, the apparatus for managing the digital access elements, or the single combined apparatus, would not comprise the digital access element storage for storing the digital access elements. Moreover, the retrieval data storage would store retrieval data associated with the digital access elements, wherein now the retrieval data would allow not, i.e. not directly, for a retrieval of the digital access elements from a digital access element storage, but for generating digital access element based thereon, particularly for recovering digital identifiers, access data and/or coating material data. The data stored on the retrieval data storage may be stored according to digital identifiers associated with the data, wherein the digital identifiers indicate respective coating materials to which the respective stored retrieval data relate. Hence, retrieval data relating to a coating material with a given digital identifier may be retrieved from the retrieval data storage based on the digital identifier.

The stored retrieval data may comprise the digital identifier(s) and associated access data. For instance, based on the decentral identifier, the associated access data may be retrieved. The stored retrieval data may comprise representations of digital identifiers, particularly hash values determined based on digital identifiers. The stored retrieval data may comprise digital identifier(s) and associated data related to the coating material, such as a physical identifier. For instance, based on the physical identifier, the associated decentral identifier may be retrieved. The retrieval data may comprise a concatenation generated from the digital identifier associated with the coating material and the precursor digital identifier(s) based on a relationship representation as previously described. For instance, the concatenation may be represented by a hash value.

The retrieval data storage may correspond to a distributed ledger or any other registry. The retrieval data storage may correspond to a database. Since the retrieval data may contain authentication information associated with digital access elements and/or digital identifiers contained in digital access elements, the retrieval data store may be regarded as an authentication registry. The retrieval data store may be configured to resolve the concatenation contained in the retrieval data. For instance, the retrieval data store may be configured to resolve, based on the relationship representation, the concatenation and to provide data on the precursor materials used to produce the coating material. Said data may include the precursor material digital identifiers. Hence, the retrieval data store may allow to determine the precursor materials used to produce the coating material. Moreover, the retrieval data store may allow to determine the materials used to produce a component, a component assembly or an end product, facilitating track and trace of materials along the entire value chain.

In a further aspect an apparatus for generating a digital access element associated with a coating material is presented, the apparatus comprising: one or more computing nodes; and one or more computer-readable media having thereon computer-executable instructions that are structured such that, when executed by the one or more computing nodes, cause the apparatus to perform the following steps:
receiving a request to provide a digital identifier associated with a data owner and coating material data, particularly wherein the data owner controls access to the coating material data, e.g. by a data consuming service
in response to the request, providing the decentral identifier and generating the digital access element including the digital identifier and access data;
providing the digital access element for access by a data consuming service under control or controlled by a data providing service associated with the data owner, particularly wherein the data providing service comprises computer-executable instructions for providing and/or processing coating material data associated with the data owner e.g. for accessing and/or processing by the data consuming service.

In one aspect an apparatus for generating a digital access element associated with a coating material is presented, the apparatus comprising: one or more computing nodes; and one or more computer-readable media having thereon computer-executable instructions that are structured such that, when executed by the one or more computing nodes, cause the apparatus to perform the following steps:
providing a digital identifier associated with a data owner and coating material data, particularly wherein the data owner controls access to the coating material data, e.g. by a data consuming service
generating the digital access element including the digital identifier and access data;
providing the digital access element for access by a data consuming service under control or controlled by a data providing service associated with a data owner, particularly wherein the data providing service comprises computer-executable instructions for providing and/or processing coating material data associated with the data owner e.g. for accessing and/or processing by the data consuming service.

In a further aspect, the invention relates to a method for generating a digital access element associated with a coating material, wherein the method includes a) providing a digital identifier associated with the coating material and coating material data, and b) generating the digital access element based on the digital identifier and access data.

In another aspect disclosed is a computer-implemented method for generating a digital access element associated with a coating material, said digital access element including a digital identifier and access data, particularly a digital representation pointing to coating material data associated with the coating material, the method comprising the steps:
receiving a request to provide the digital identifier associated with coating material data and a data owner, particularly wherein the data owner controls access to the coating material data, e.g. by a data consuming service;
in response to the request, providing the decentral identifier and generating the digital access element including the digital identifier and the access data;
providing the digital access element for access by the data consuming service controlled by or under control by a data providing service associated with the data owner, particularly wherein the data providing service comprises computer-executable instructions for providing and/or processing coating material data associated with the data owner e.g. for accessing and/or processing by the data consuming service.

In another aspect disclosed is a computer-implemented method for generating a digital access element associated with a coating material, said digital access element including a digital identifier and access data, particularly a digital representation pointing to coating material data associated with the coating material, the method comprising the steps:
providing a digital identifier associated with a data owner and coating material data, particularly wherein the data owner controls access to the coating material data, e.g. by a data consuming service
generating the digital access element including the digital identifier and access data;
providing the digital access element for access by a data consuming service under control or controlled by a data providing service associated with a data owner, particularly wherein the data providing service comprises computer-executable instructions for providing and/or processing coating material data associated with the data owner e.g. for accessing and/or processing by the data consuming service.

In yet another aspect disclosed is a computer-implemented method for using a digital access element, preferably to further process the coating material associated with the digital access element, the method comprising the steps:
receiving a request to access coating material data associated with a digital identifier of the digital access element as generated according to the methods disclosed herein or by the apparatuses disclosed herein;
optionally authenticating and/or authorizing the request to access the coating material data;
based on optionally the authentication and/or authorization, providing access to the coating material data associated with the digital identifier of the digital access element.

In a further aspect, the invention relates to a computer program for generating a digital access element associated with a coating material, wherein the program comprises instructions which, when the program is executed by one or more computers, cause the one or more computers to execute the above method. The one or more computers can realize a distributed computing environment in which the program can be executed.

In a further aspect, the invention relates to a use of the digital access element as generated by the above apparatus or according to the above method during the manufacturing of products using a coating material associated with the digital access element.

In a further aspect, the invention relates to a coating material associated with a digital access element generated by the above apparatus or according to the above method.

In yet another aspect a system including a coating material associated with a digital access element is disclosed, wherein the digital access element including a digital identifier and access data is generated for the coating material according to the methods or by the apparatuses lined out herein.

In yet another aspect a digital access element including a digital identifier and access data is disclosed, wherein the digital access element is generated for the coating material according to the methods or by the apparatuses lined out herein.

It shall be understood that the aspects described above, specifically the apparatus of claim 1, the method of claim 14 and the computer program of claim 15, have similar and/or identical preferred embodiments, in particular as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an apparatus for generating a digital access element associated with a coating material,

FIG. 2 shows schematically and exemplarily an apparatus for manufacturing a coating material,

FIG. 3 shows schematically and exemplarily a relation of a digital access element to further data structures,

FIG. 4 shows schematically and exemplarily a further relation of a digital access element to further data structures,

FIGS. 5-10 show schematically and exemplarily possible relations between digital access elements,

FIGS. 11A, 11B illustrate examples of relationship representations which may be used for generating a concatenation,

FIG. 12 shows an example of a system for producing a coating material associated with a digital access element including an example method for generating the digital access element, and

FIG. 13 shows a schematic illustration of providing access, via a data providing service associated with a data owner, to a digital access element associated with a coating material using a data consuming service associated with a data user.

### DETAILED DESCRIPTION OF EMBODIMENTS

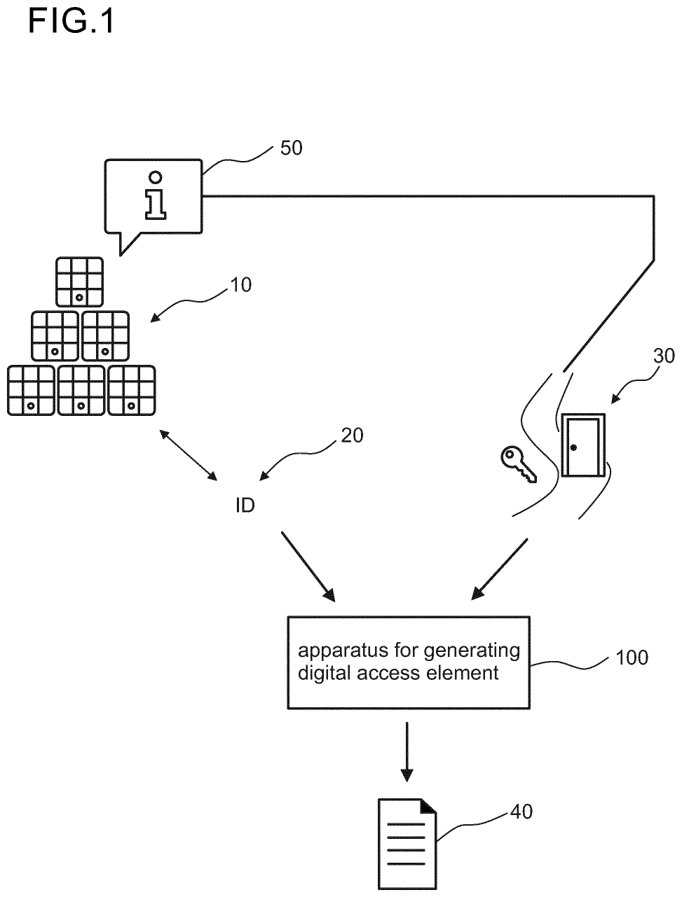

FIG. 1 shows schematically and exemplarily an apparatus 100 for generating a digital access element 40 associated with a coating material 10 in action. While not explicitly indicated in FIG. 1, the apparatus 100 comprises a digital identifier providing unit configured to provide a digital identifier 20 associated with the coating material 10 and coating material data 50, and a digital access element generator configured to generate the digital access element 40 based on the digital identifier 20 and access data 30. The coating material 10 is shown in terms of a batch of manufactured material, wherein the material batch has been filled into several separate containers.

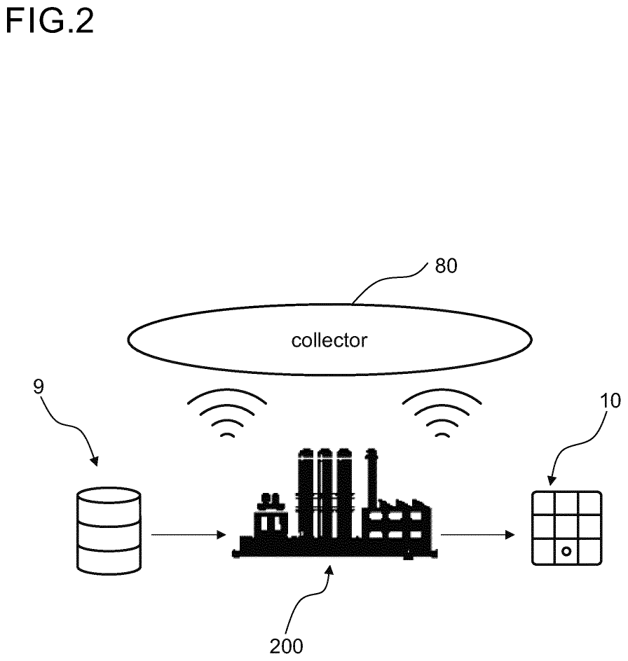

While not being shown in FIG. 1, identifying elements have been attached to each of the containers, wherein the identifying elements correspond to physical identifiers for the batch of coating material 10. The digital identifier providing unit may be configured to provide the digital identifier 20, which is associated with the batch of coating material 10 and thereby also to the coating material data 50, based on any of the physical identifiers. For instance, the digital identifier may be provided by scanning RFID tags serving as identifying elements attached to the containers of the coating material 10. The coating material data 50 may be collected during manufacturing of the batch of coating material 10, but could also be collected prior to the actual manufacturing process or afterwards. For instance, the coating material data 50 could be indicative of data relating to precursor materials 9 used in the manufacturing of the coating material 10 as indicated in FIG. 2, including transport data of the precursor materials 9, or to a further transport of the manufactured coating material 10.

As illustrated schematically and exemplarily by FIG. 2, for collecting the coating material data 50, the apparatus 100 may comprise a collector 80 which is configured to collect the coating material data 50, wherein the digital identifier 20 provided by the digital identifier providing unit may be associated with the collected coating material data 50. As also indicated by FIG. 2, the coating material data 50 may be collected by the collector 80 from a manufacturing apparatus 200 at a manufacturing site at which the coating material 10 is manufactured from one or more precursor materials 9, wherein the collector 80 may, for instance, wirelessly receive manufacturing and/or transport data from data acquisition devices at a plurality of locations in and around the manufacturing site.

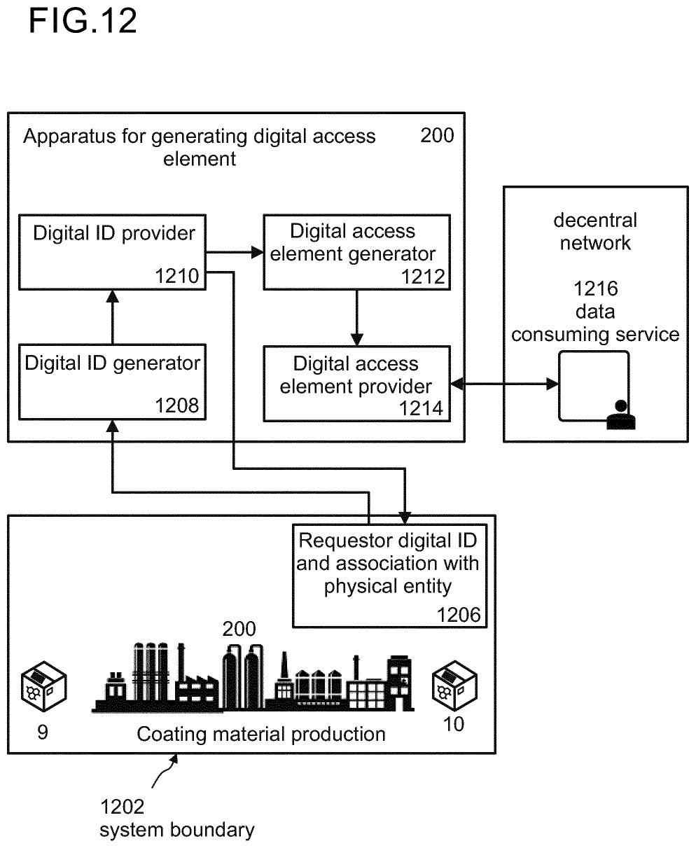

The access data 30 can refer to any data needed for accessing the coating material data 50. Hence, the type of access data 30 can vary significantly between embodiments. Examples of access data 30 include a storage place or digital address of the coating material data 50, data needed in or indicative of an authentication mechanism and/or an authorization mechanism to be carried out before being able to access the coating material data 50, and data indicative of data providing services responsible for providing the coating material data 50. For instance, the apparatus may be configured to generate the digital access element 40 upon receiving a request to provide at least the digital identifier 20. The request may be associated with the chemical production 200 producing the coating material 10. For instance, the request may be generated by the computing system of the chemical production 200 upon detection of the physical identifier on each packaging unit of the produced coating material 10 as described in the context of FIG. 12. The request may particularly be directed to the digital identifier providing unit, which may be configured to provide the requested digital identifier 20 upon receipt of the request, wherein the digital access element generator may be configured to generate the digital access element 40 based on the requested digital identifier 20 and access data 30. The digital identifier providing unit be configured to provide—apart from the digital identifier associated with the coating material and the coating material data (hereinafter referred to as first digital identifier)—at least one precursor digital identifier associated with one or more precursor materials from which the coating material is produced. The digital identifier providing unit may be configured to generate a concatenation of the first digital identifier and precursor identifier(s) based on a relationship representation according to which the first digital identifier is associated with the precursor identifier(s). The relationship representation may be associated with a relationship between the coating material and each precursor material. The relationship representation may specify that the precursor material may be used to produce the coating material and/or the coating material may be produced by using the precursor material. Hence, how the digital identifiers of the coating material and the precursor materials are concatenated may depend, for instance, on a relation between the coating material and the precursor materials in the supply chain and/or to how the coating material is produced from the precursor materials. Moreover, if the precursor materials relate to several stages in the supply chain, such that some of the precursor materials are themselves used in the production of other precursor materials, concatenation may be performed for one or more stages. For instance, each of the precursor digital identifiers may be concatenated with the digital identifier of the coating material. Alternatively, each of the precursor digital identifiers may be concatenated with the digital identifier associated with the intermediate product produced from precursor materials associated with said digital identifiers, and the resulting concatenation may be concatenated with the digital identifier of the coating material. One or more hash value(s) may be generated based on the relationship representation. Hash value(s) may be generated based on the concatenation of digital identifiers. Hash value(s) may be generated for the concatenation of digital identifiers. Hash value(s) may signify the concatenation of digital identifiers. The hash values may be generated based on data associated with a first digital identifier and the at least one precursor digital identifier. The hash value may be generated based on a combined data set associated with the first digital identifier and the precursor material digital identifiers. By concatenating digital identifiers associated with products of the chemical product supply chain and/or the recycling product chain, the physical entities can be tracked and traced virtually. The access data 30 may indicate how the coating material data 50 associated with a coating material 10 having the specific digital identifier 20 may be accessed. Hence, the type of access data can vary significantly between embodiments. Examples of access data include digital representation(s) pointing to the coating material data or parts thereof 50. Such digital representation(s) may include endpoint addresses of data providing services associated with the coating material data, e.g. having access to data structures storing the respective coating material data or parts thereof 50. Access data may include data needed in or indicative of an authentication mechanism and/or an authorization mechanism to be carried out before being able to access the coating material data 50.

The digital access element 40 generated by the apparatus 100 may be a digital document or other digital data structure including the digital identifier 20 and the access data 30. It should be emphasized already, however, that the digital access element 40 may also include a part of the coating material data 50 itself. Hence, some of the coating material data 50 may be included in the generated digital access element 40 and thereby directly provided to a data consumer, such as a coating material recipient, while other of the coating material data 50, which may be more sensitive and therefore more security relevant data, may not be included in the generated digital access element 40, but only be accessible based on the digital access element 40 upon conditions to be met by, for instance, the data consuming service associated with the data consumer.

The coating material 10 may be manufactured using a plurality of precursor materials. The precursor materials may refer to materials acquired or manufactured earlier than the coating material in a supply chain of the coating material. Also to the plurality of precursor materials respective digital identifiers may be associated, which can be referred to as precursor digital identifiers. The digital access element generator of the apparatus 100 may be configured to generate the digital access element 40 based on a combined digital identifier, determined based on the digital identifier 20 associated with the coating material 10 and the one or more precursor digital identifiers, i.e. the digital identifiers of the precursor materials. The combined digital identifier may be determined based on the digital identifier 20 associated with the coating material 10 and the plurality of precursor digital identifiers in accordance with a relationship representation. The relationship representation may be associated with a relationship between the coating material 10 and each precursor material. The relationship representation may, for example, include a hash chain, such as a hash chain produced from hashing precursor material data, such as the digital identifier and/or data of the precursor material, and hashing coating material data, such as the digital identifier and/or data of the coating material itself.

Hence, how the digital identifiers of the coating material 10 and the precursor materials are combined to the combined digital identifier may depend, for instance, on a relation between the coating material 10 and the precursor materials in the supply chain and/or to how the coating material 10 is manufactured from the precursor materials. Moreover, if the precursor materials relate to several stages in the supply chain, such that some of the precursor materials are themselves used in manufacturing of other precursor materials, a combined digital identifier based on which the digital access element for the coating material is generated may be determined in terms of different types of combinations of the precursor digital identifiers. For instance, the combined digital identifier may be determined as a function of each of the precursor digital identifiers. Alternatively, each of the precursor digital identifiers may itself be determined as a function of a digital identifier associated with a material used in producing the respective precursor material, and the combined digital identifier may be determined as a function of only the immediately preceding precursor digital identifier, such that it depends only indirectly on the precursor digital identifiers earlier in the supply chain, namely via the immediately preceding precursor digital identifier.

The coating material data 50 may include data on the plurality of precursor materials, the manufactured coating material itself, a transport of the precursor materials to a manufacturing side of the coating material 10, and/or a further transport of the manufactured coating material 10. As mentioned above, some of these data 50 may only be accessible based on the generated digital access element 40 upon meeting pre-defined conditions, while others may be directly included in the digital access element 40 and hence immediately accessible to a data consuming service associated with a data consumer, such as the coating material recipient.

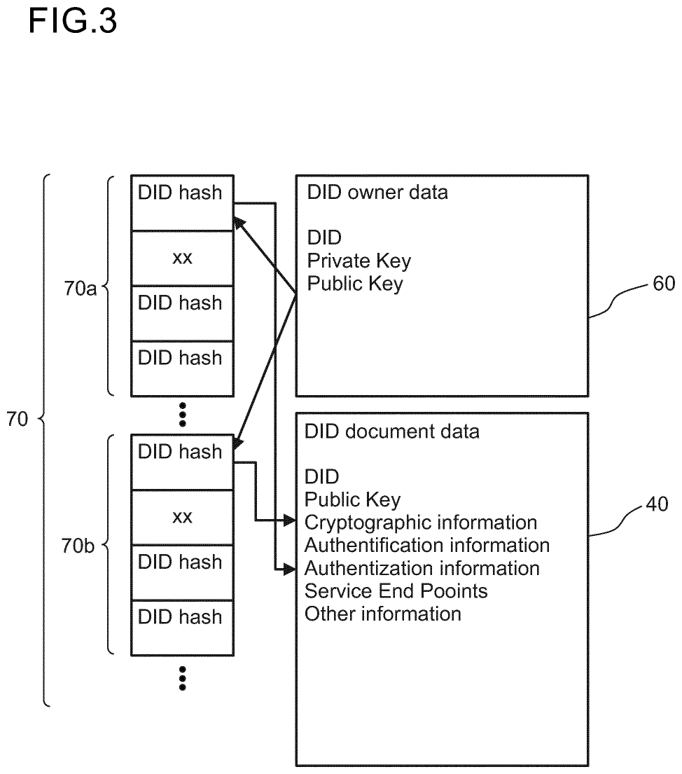

FIG. 3 shows schematically and exemplarily a relation of a digital access element 40 for a coating material 10 to further data structures in an embodiment.

The digital identifier 20, which by itself could be abbreviated ID, is in this case a decentralized ID and abbreviated by DID. Moreover, the ID-based digital access element 40 could in this case be a DID document associated with the DID.

Besides the DID document serving as digital access element 40, FIG. 3 shows a DID owner data element 60 including ID-based owner data. Generally, the ID-based owner data may include an ID associated with a subject such as coating material data 50 and may include authentication mechanisms. Moreover, the ID-based owner data may include owner data that is electronically owned and controlled by the DID owner. In this context “electronically owned” may refer to data that is stored in an owner repository or wallet. Such data may be securely stored and/or managed on an organizational server or client device. In FIG. 3 it is just shown that the ID-based owner data may include the DID, a private key and a public key. The ID-based owner may own and control the DID, which represents an identity associated with the DID subject, a private key and public key pair that are associated with the DID. The DID may be understood as including an identifier and also authentication information associated with or uniquely linked to the identifier.

The DID subject may be a precursor material like a raw material or basic substance used in manufacturing the coating material 10, the coating material 10 itself, a component of any of the foregoing, a component assembly, an end product in which the coating material is used, or a transport of a precursor material or the coating material 10. The DID subject may be also a machine, a system, or a device used for manufacturing or transporting any of the precursor material, the coating material 10 or the end product, or a collection of such machine(s), device(s) and/or system(s). The DID owner may be a supply chain participant or a manufacturer such as a coating material producer. The DID owner may be an upstream participant in the supply chain of the coating manufacturer such as a supplier that supplies raw chemical products or precursors to produce coating materials. The DID owner may be a downstream participant in the supply chain of the coating manufacturer such as a customer that consumes chemicals to produce the coating materials. The DID owner may be any participant of the supply chain, including a raw chemical product supplier, intermediate chemical products manufacturer, intermediate part manufacturer, component manufacturer, component assembly manufacturer or end product manufacturer.

The DID may be any identifier that is associated with the DID subject and the DID owner. Preferably, the DID is unique to the DID subject and/or DID owner. The DID may be unique at least within the scope in which the DID is anticipated to be in use. The DID may be a locally or globally unique identifier for any of the above mentioned possible DID subjects. The DID may also be a Uniform Resource Identifier (URI) such as a Uniform Resource Locator (URL). Moreover, the DID may be an Internationalized Resource Identifier (IRI). Concretely, the DID may be a random string of numbers and letters for increased security. In one embodiment, the DID may be a string of 128 letters and numbers. The DID may be decentralized, independent of a centralized, third-party management system and under the control of the DID owner.

The digital access element 40 as DID document is associated with the DID, i.e. the DID included in the ID-based owner data. Accordingly, the digital access element 40 may include a reference to the DID, which is associated with the DID subject that is described by the DID document. As shown in FIG. 3, the DID document may include, as access data 30, data corresponding to authentication, or authorization, information such as, or in addition to, a public key. The public key may be used by third-party entities that are given permission by the DID owner/subject to access information and data owned by the DID owner/subject, particularly any coating material data 50 not already included in the DID document 40. The public key may also be used for verifying that the DID owner, in fact, owns or controls the DID. As shown in FIG. 3, the DID document may include, as further access data 30, (further) cryptographic information and authorization information, e.g. to authorize third party entities to read the DID document or some part of the DID document, particularly without giving the third party the right to prove ownership of the DID.

The digital access element 40 may also include one or more representations that digitally link to the coating material data 50, e.g. by way of service endpoints. A service endpoint may include a network address at which a service operates on behalf of the DID owner. In particular, the service endpoints may refer to services of the DID owner that give access to coating material data 50. Such services may include services to read or analyse the coating material data 50. Additionally or alternatively to the possible types of coating material data 50 indicated already, the coating material data 50 may include declaration data, safety data, certificate of analysis data, emission data, carbon footprint data, environmental footprint data, product specification data, product information, technical application data, manufacturing data, transport data or combinations thereof. The digital access element 40 may further include various other information, such as metadata specifying when the digital access element 40 was generated, when it was last modified and/or when it expires.

As shown in FIG. 3, the DID and the digital access element 40 may be associated with a retrieval data storage 70 in the form of a data registry, which may be provided by a centralized data service system or a decentralized data service system, e.g. a distributed ledger, a blockchain or a distributed file system. Possible blockchain systems include Quorum and Hyperledger Fabric. The distributed ledger, blockchain or distributed file system may be used to store a representation of the DID that points to the digital access element 40. A representation of the DID may be stored on distributed computing nodes 70a, 70b of the distributed ledger, distributed file system or blockchain. For example, a DID hash may be stored on multiple computing nodes 70a, 70b of the distributed ledger and point to the location of the digital access element 40. Each of the computing nodes 70a, 70b may store a copy of the distributed ledger. In this way, each DID hash can be stored redundantly, thereby allowing for an increased data safety. DIDs associated with a plurality of different digital access elements 40 and/or coating materials 50 may be included in the registry 70.

In some embodiments, the digital access element 40 may be stored on the distributed ledger, i.e. either additionally or alternatively to the associated DID representation being stored on the distributed ledger. In other embodiments, the digital access element 40 may be stored in a data storage (not illustrated) that is associated with the distributed ledger or blockchain.

The distributed ledger or blockchain may be any decentralized, distributed network that includes various computing nodes that are in communication with each other. For example, the distributed ledger may include a first distributed computing node, a second distributed computing node, a third distributed computing node, and any number of additional distributed computing node. The distributed ledger or blockchain may operate according to any known standards or methods for distributed ledgers. Examples of conventional distributed ledgers that correspond to the used distributed ledger or blockchain include, but are not limited to, Bitcoin [BTC], Ethereum, and Litecoin.

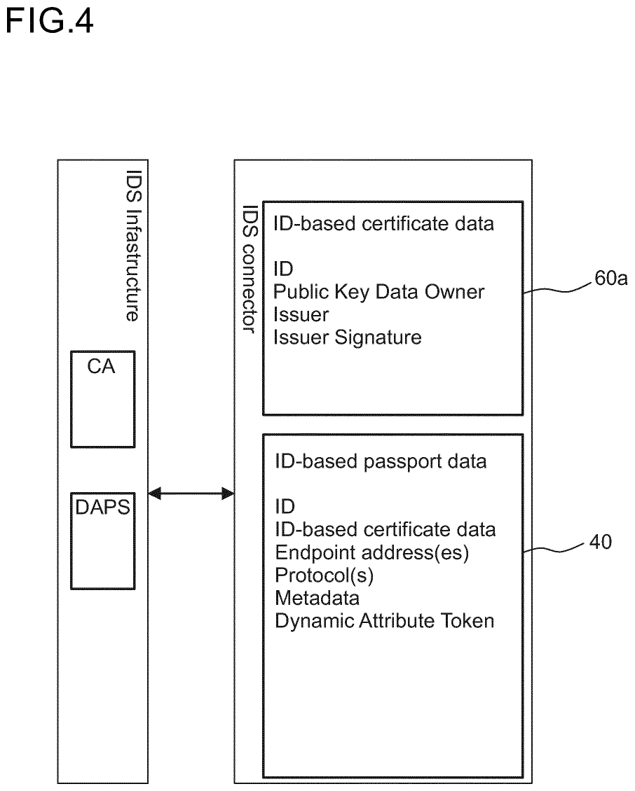

FIG. 4 shows schematically and exemplarily a relation of a digital access element 40 for a coating material 10 to further data structures in a further embodiment

In contrast to the example of FIG. 3, the example of FIG. 4 is certificate-based, i.e. based on a certificate 60a instead of a data structure like a DID owner data element 60. The certificate 60a includes in this case ID-based certificate data, specifically authentication data of the certificate owner and the certificate issuer. For example, a cryptographic signature from the issuer may bind the public key of the data owner to the ID. The ID may be a unique ID (such as a UID) as described in relation to the DID of FIG. 3. The certificate may be a X.509 certificate such as X509v3. The digital access element 40 is in this case an ID-based passport including ID-based passport data, which may be associated with a data source of the data owner. The ID-based passport data may include an ID, authentication data and endpoints associated with the coating material data 50. Such endpoints may include any digital representation connecting to the data source. The data source may provide the coating material data 50.

In this certificate-based example, the ID-based passport data includes one or more certificate(s) associated with the data owner. The certificates may be associated with an identity manager including, e.g., a certificate issuing service and/or a dynamic provisioning service providing dynamic attribute tokens (e.g. OAuth Access Tokens). The information required to verify the certificates are provided via an authentication registry associated with the certificate issuing service and/or a dynamic provisioning service. For instance, in the IDSA

Reference Architecture Model, Version 3.0 of April 2019, a connector associated with the data owner, a Certification Authority (CA), a Dynamic Attribute Provisioning Service (DAPS) and a connector associated with the data consumer service are used to verify the identity prior to performing a data exchange (not shown). For this purpose, such connectors include one or more certificate(s) such as X.509 certificate(s). This way the connector possesses a unique identifier embedded in a X.509 certificate that identifies the connector instance.

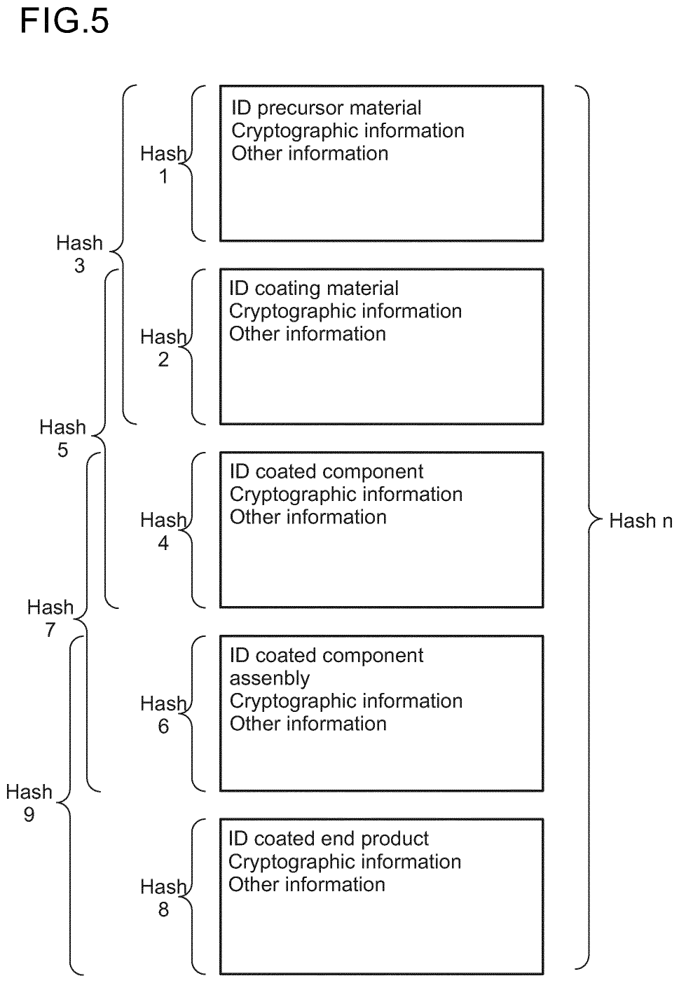

FIGS. 5-10 show different example configurations for digital access elements anchored by digital identifiers (IDs). The configurations include different parent, child, grandchild and so on relationships for digital access elements generated in the supply chain involving coating material production, up to an end product manufactured using components coated with the coating material. Since the coating material is chemically produced and economic value is generated along the supply chain, the supply chain is subsequently referred to as a chemical value chain.

FIG. 5 illustrates what could be referred to as an “individual” configuration for different digital access elements generated in the chemical value chain. For multiple product stages in the chemical value chain individual digital access elements may be generated, among them a digital access element 40 associated with a coating material 10. The digital access element generation may include the providing of a digital, particularly decentral, identifier and access data indicating a required authentication mechanism or service endpoint to access the coating material data 50 for each of the multiple product stages. The digital access elements for the multiple product stages may be based on crypto signatures as part of the access data. For instance, the digital access elements for the multiple product stages may be concatenated through hash values based on different data sets, specifically the different data included in the respective digital access elements. As shown in FIG. 5, hash 1 may be based on data of a digital access element associated with a precursor material (“precursor material digital access element”), hash 2 may be based on data of the digital access element associated with the coating material (“coating material digital access element”) and hash 3 may be based on data of the raw material digital access element in combination with, particularly concatenated with, data of the coating material digital access element 40. The hashes may be used to generate a hash chain allowing to determine the materials used to produce a coating material, a coated component, a coated component assembly and/or a coated end product. Further combination and particularly concatenation may be done for other combinations of digital access elements up to hash n, which combines, particularly concatenates, digital access elements up to the digital access element associated with an end product (“end product digital access element”). The sequence of hashes from hash 1 to hash n can be viewed as a “mirror” of the value chain from the raw material to the coated end product, since it reflects the relationships between the respective digital access elements. Concatenation via hashes of the crypto signature is only one example concatenation. Other examples include permission aggregations with different scope of data that may be embedded in child passports, public key aggregations with different crypto signatures or service endpoint aggregation with different links.

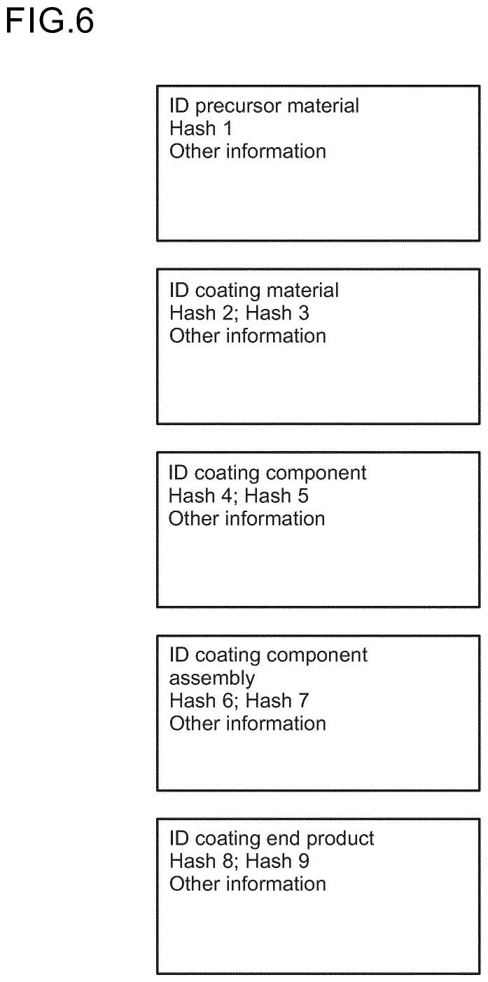

FIG. 6 illustrates different digital access elements including a concatenation associated with multiple digital identifiers based on the relationship representation for different products associated with product stages of the chemical value chain. Among the digital access elements, one digital access element is associated with a coating material. In this particular example, hash values are used for the concatenation associated with multiple digital identifiers. The data sets to generate the hash values are schematically illustrated in FIG. 5.

The precursor material digital access element may be provided to the coating material producer using the precursor material to produce the coating material. The precursor material digital access element may be connected to a hash value hash 1. The hash value hash 1 may be generated via a hashing algorithm such as MD5, SHA-1, SHA-2, SHA-3 or any other suitable algorithm based on a one-way function that can't be reverse engineered. The hash value hash 1 may be generated based on data included in or connected to the precursor material digital access element. The data for hash generation may include the digital identifier and the access data. The data for hash generation may include the precursor material data. The data for hash generation may include the digital identifier associated with the precursor material, the access data, the precursor material data associated with the precursor material and/or cryptographic information connected to the digital identifier. The hash value hash 1 may be used by participant nodes of the chemical supply chain to check integrity of the data package transferred from the precursor material supplier e.g. to the coating material producer.

Similar to the precursor material digital access element, the coating material digital access element may be provided to the component producer using the coating material to produce a component, such as a coated substrate. The generation of the coating material digital access element may be based on the precursor material digital access element provided to the coating material producer using the precursor material to produce the coating material. The coating material digital access element may be connected to one or more hash value(s) hash 2, hash 3. The hash values hash 2, hash 3 may be generated via a hashing algorithm such as MD5, SHA-1, SHA-2, SHA-3 or any other suitable algorithm based on a one-way function that can't be reverse engineered. The hash values hash 2, hash 3 may be generated based on data included in or connected to the coating material digital access element and/or the precursor material digital access element. The hash value(s) hash 2, hash 3 may be generated based on clear data itself or based on hash value(s) generated from the clear data. For instance, hash 3 may be generated based on digital access element data or based on hashed digital access element data. The data for hash generation may include the digital identifier associated with the precursor material used to produce the coating material, the digital identifier associated with the coating material, the access data associated with the precursor material digital access element and/or the access data associated with the coating material digital access element.

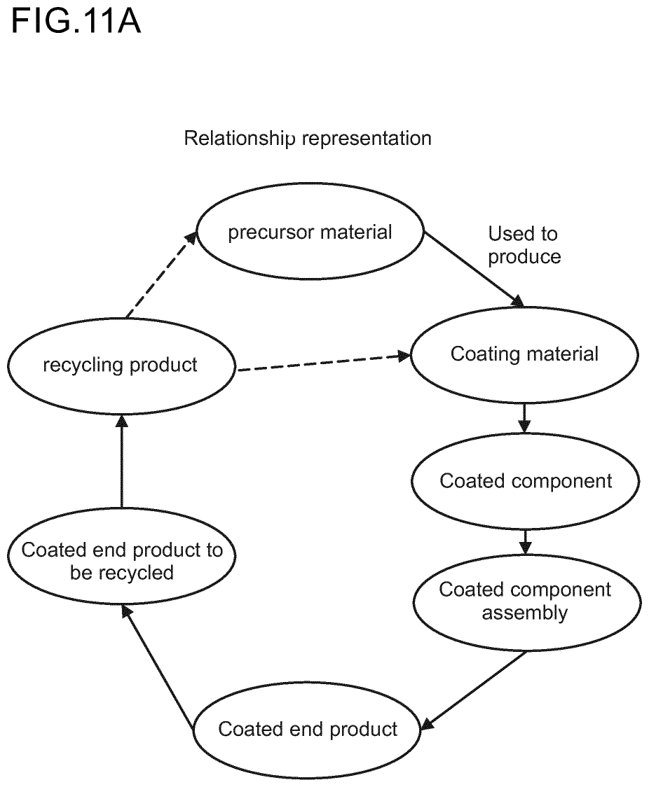

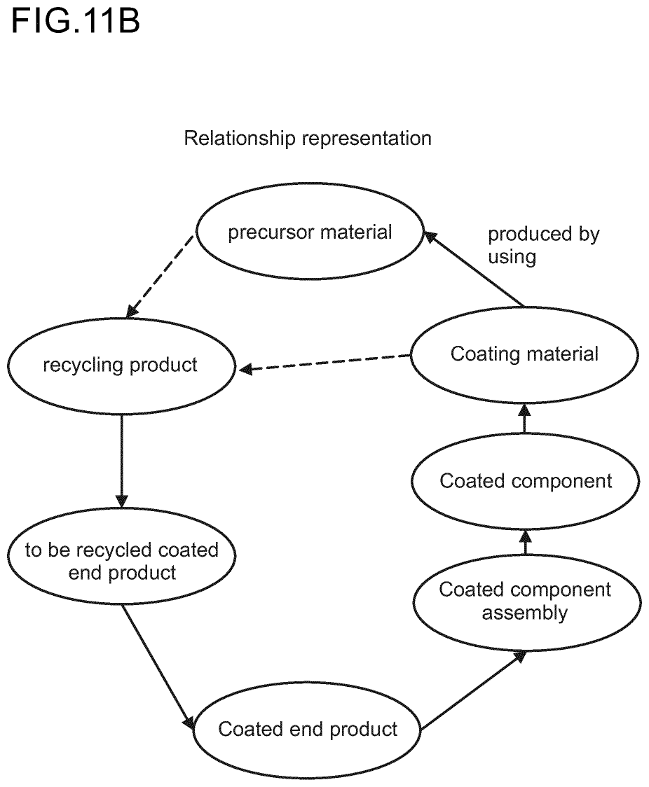

The concatenation associated with multiple digital identifiers may relate to the digital identifiers associated with the coating material and the precursor material(s). Hashing data related or included in the respective digital access elements may provide for such concatenation. The data for hash generation may include the precursor material data and/or the coating material data. The data for hash generation may include the digital identifier associated with the precursor material, the access data, the precursor material data associated with the precursor material and/or cryptographic information connected to the digital identifier. The data for hash generation may include the digital identifier associated with the coating material, the access data, the coating material data associated with the coating material and/or cryptographic information connected to the digital identifier. Hash value hash 2 may be generated in relation to the coating material digital access element as illustrated in FIG. 11. The hash value hash 2 may be based on at least the digital identifier associated with the coating material and the access data. Hash value hash 3 may be generated in relation to the precursor material digital access element and the coating material digital access element as illustrated in FIG. 5. The hash value hash 3 may be based on at least the digital identifier associated with the precursor material, the access data associated with the precursor material digital access element, the digital identifier associated with the coating material and the access data associated with the coating material digital access element. The hash value(s) may be used by participant nodes of the chemical supply chain to check integrity of the data package transferred from the precursor material supplier or the coating material producer. The combined hash value(s) may further be used by participant nodes of the chemical supply chain to determine the relation of products at different stages and to check integrity of such relation.

As illustrated in FIGS. 5 and 6, the hash value(s) may be connected to the digital access elements associated with one or more product stage(s) of the chemical supply chain.

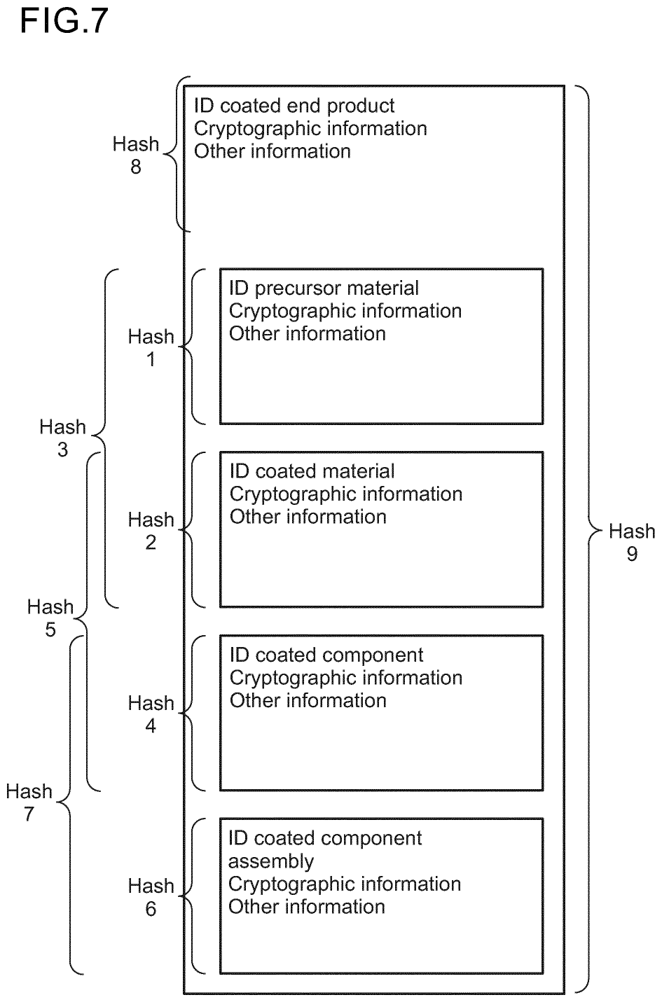

FIG. 7 illustrates what could be referred to as an “anchored” configuration for different digital access elements generated in the chemical value chain. For the end product an end product digital access element is generated. For multiple further product stages in the chemical value chain individual digital access elements may be generated, among them the coating material digital access element 40, wherein the individual digital access elements are embedded in or linked with the end product digital access elements. The digital access element generation may include the providing of a decentral identifier and access data indicating a required authentication mechanism and/or service endpoint to access the coating material data 50 for each of the multiple product stages. The digital access elements for the multiple product stages may be based on crypto signatures as part of the access data. For instance, the digital access elements for the multiple further product stages may be concatenated through hash values based on different data sets, specifically the different data included in the respective digital access elements. As shown in FIG. 7, hash 1 may be based on data of the precursor material digital access element, hash 2 may be based on data of the coating material digital access element 40 and hash 3 may be based on data of the precursor material digital access element in combination with, particularly concatenated with, data of the coating material digital access element 40. Further combination and particularly concatenation may be done for other combinations of product passports up to hash n, which combines/concatenates product passports up to the end product passport. Concatenation via hashes of the crypto signature is only one example concatenation. Other examples include permission aggregations with different scope of data that may be embedded in child passports, public key aggregations with different crypto signatures or service endpoint aggregation with different links.

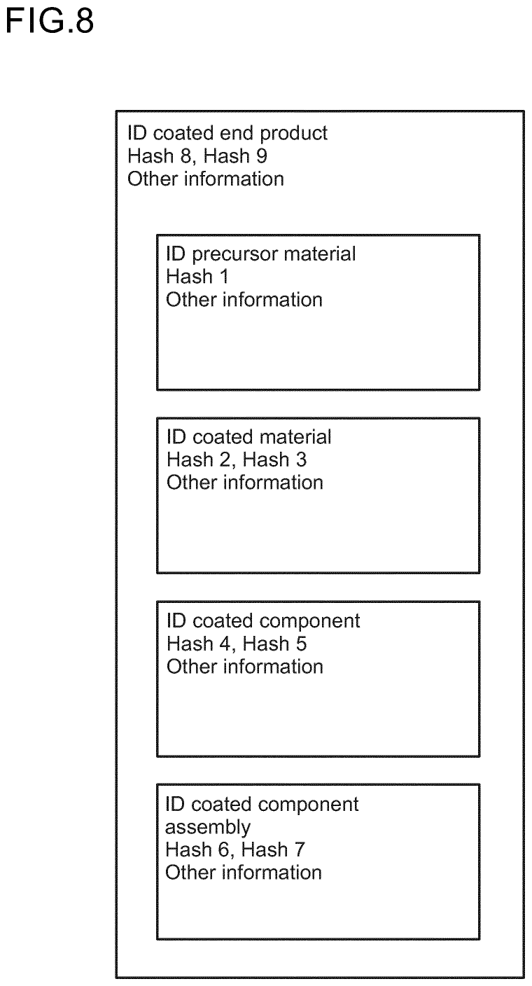

FIG. 8 illustrates different digital access elements including a concatenation associated with multiple digital identifiers based on a relationship representation for different products associated with product stages of the chemical value chain. Among the digital access elements, one digital access element is associated with a coating material. In this particular example, hash values are used for the concatenation associated with multiple digital identifiers. The data sets to generate the hash values are schematically illustrated in FIG. 7.

The precursor material digital access element may be provided to the coating material producer using the precursor material to produce the coating material. The precursor material digital access element may be connected to a hash value hash 1 that may be generated as described in the context of FIG. 7.

Similar to the precursor material digital access element, the coating material digital access element may be provided to the component producer using the coating material to produce a component, such as a coated substrate. The hash values may be generated as described in the context of FIG. 6. The hash values may be generated in a similar fashion for the digital access element up to the end product digital access element.

In the anchored configuration, the end product digital access element may include hash values that relate to the digital access element associated with the products used to produce the end product. The concatenation associated with multiple digital identifiers may in this case relate to the digital identifiers associated with the precursor materials(s), the coating material(s), the component(s) and the component assembly/ies. Hashing data related or included in the respective digital access elements may provide for the concatenation. The data for hash generation may include any data included in respective digital access elements. The hash value hash 7 may be based on at least the digital identifier associated with the end product and access data associated with the end product digital access element. Hash value hash 8 may be generated in relation to the product digital access element associated with products at different product stages as illustrated in FIG. 8. The combined hash value hash 8 may be used by participant nodes of the chemical supply chain to determine the relation of products at different stages and to check integrity of such relation.

As illustrated in FIGS. 7 and 8, the hash value(s) may be connected to the digital access elements associated with one or more product stage(s) of the chemical supply chain.

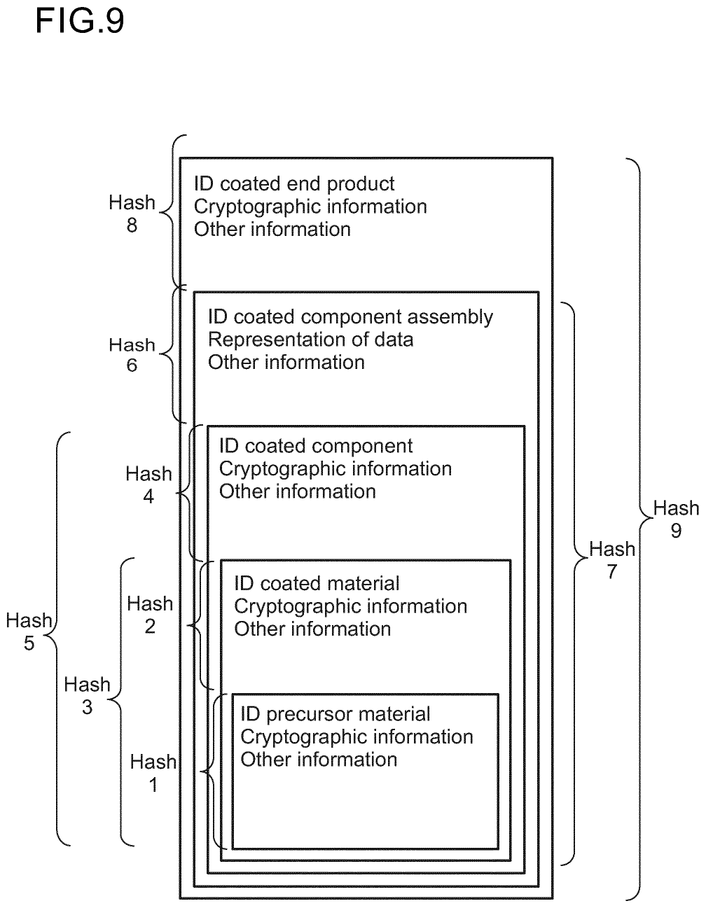

FIG. 9 illustrates what could be referred to as a “fully embedded” configuration for different digital access elements generated in the chemical value chain. For multiple product stages in the chemical value chain individual digital access elements may be generated, among them the coating material digital access element 40. The digital access element generation may include the providing of a decentral identifier and access data indicating a required authentication mechanism and/or service endpoint to access the coating material data 50 for each of the multiple product stages. The digital access elements for the multiple product stages may be based on crypto signatures as part of the access data. For instance, the product passports for the multiple product stages may be concatenated through hash values based on different data sets, specifically the different data included in the respective digital access elements. As shown in FIG. 9, hash 1 may be based on data of the raw material digital access element, and hash 2 may be based on data of the raw material digital access element in combination with, particularly concatenated with, data of the coating material digital access element 40. Further concatenation may be done for other combinations of product passports up to hash n, which concatenates digital access elements up to the end product digital access elements. Concatenation via hashes of the crypto signature is only one example concatenation. Other examples include permission aggregations with different scope of data that may be embedded in child passports, public key aggregations with different crypto signatures or service endpoint aggregation with different links.

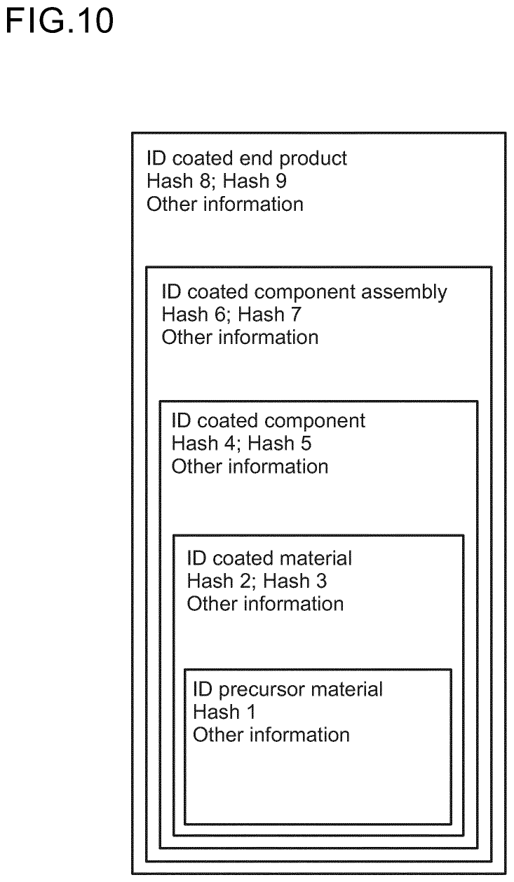

FIG. 10 illustrates different digital access elements including a concatenation associated with multiple digital identifiers based on a relationship representation for different products associated with product stages of the chemical value chain. Among the digital access elements, one digital access element is associated with a coating material. In this particular example, hash values are used for the concatenation associated with multiple digital identifiers. The data sets to generate the hash values are schematically illustrated in FIG. 15.

The precursor material digital access element may be provided to the coating material producer using the precursor material to produce the coating material. The precursor material digital access element may be connected to a hash value hash 1 that may be generated as described in the context of FIG. 15. Similar to the precursor material digital access element, the coating material digital access element may be provided to the component producer using the coating material to produce a component, such as a coated substrate. The hash values may be generated as described in the context of FIG. 9. The hash values may be generated in a similar fashion for the digital access elements up to the end product digital access element.

In the fully embedded configuration, the combined hash values may be generated from digital access elements associated with all products preceding the product. The concatenation associated with multiple digital identifiers may in this case relate to the digital identifiers associated with all preceding products, such as the precursor material(s), the coating material(s), the component(s) and the component assembly/ies. Hashing data related or included in the respective digital access element may provide for the concatenation. The data for hash generation may include any data included in respective digital access elements. The hash value hash 7 for example may be based on at least the digital identifier associated with the products up to the end product and the access data. Hash value hash 9 for example may be based on at least the digital identifier associated with the products up to and including the end product and the respective access data. The combined hash values hash, 3, hash 5, hash 7 and hash 9 may be used by participant nodes of the chemical supply chain to determine the relation of products at different stages and to check integrity of such relation. As illustrated in FIGS. 9 and 10, the hash value(s) may be connected to the digital access elements associated with one or more product stage(s) of the chemical supply chain.

The configurations shown in FIGS. 5-10 relate to digital access elements generated in the chemical value chain up to an end product. Similarly, digital access elements generated in the recycling chain from end product to recyclate(s) may be concatenated. Further similarly, the digital access elements generated in the chemical value chain up to an end product and in the recycling chain from end product to recyclate(s) may be concatenated. This way the circularity of products involving coating materials and in particular coating materials themselves may be virtually represented and tracked.

FIGS. 11A and 11B illustrate examples of relationship representations which may be used for generating a concatenation, for example the concatenation described in relation to FIGS. 5 to 10 above.

The relationship representation may relate to different stages of the chemical supply chain. The digital access elements at different stages of the chemical supply chain may be connected to the relationship representation. The relationship representation may be associated with the product produced at the respective stage of the chemical supply chain and at least one product used to produce the respective product. The relationship representation may be associated with the product produced at a respective stage of the chemical supply chain and at least one product produced at a previous stage of the chemical supply chain. The relationship representation may specify the relation between physical entities. The relationship representation may specify that the second physical entity may be used to produce the first physical entity as illustrated in FIG. 11A and/or that the first physical entity may be produced by using the second physical entity as illustrated in FIG. 11B. The relationship representation may relate to at least one precursor material used to produce the coating material. The relationship representation may relate to the coating material produced by using at least one precursor material.

FIG. 12 shows an example of a system for producing a coating material 10 associated with a digital access element 40, i.e. an example of the above apparatus 200, including an example method for generating the digital access element 40.

One or more raw materials and/or precursor materials 9 (hereinafter referred to as “inbound material(s)”) may be provided to the coating material production 200. The coating material production 200 may be associated with an operating system 808. The operating system may contain the apparatus described above, e.g. an apparatus comprising a digital identifier providing unit and a digital access element generator (see for example FIG. 1). The operating system may monitor and/or control the coating material production 200 based on operating parameters of the different processes. The operating system 808 may receive production demand data associated with the production planning for the coating material production 200. The production demand data may include target capacities for products produced by the coating material production 200. The operating system 808 may further receive a bill of materials associated with intermediate product(s) and/or coating material(s) to be produced. The bill of materials may include data associated with the inbound materials used to produce the intermediate product(s) or coating material(s) (such as inbound material name, inbound material identifier, inbound material amount), process data associated with the production chain for producing the intermediate product and/or the product, and/or data associated with the intermediate product/and or product to be produced, such as product specification data or data on the amount of an intermediate product and/or a product to be produced.

Based on the received production demand data and the bill of materials, material demand data may be determined. The material demand data may include data on the amount of inbound material required to produce the target capacities of intermediate product(s) and/or coating material(s). The material demand data may include inbound material identifiers associated with inbound materials required to produce the intermediate product and/or product and data on amounts associated with the respective inbound materials. The material demand data may include one or more inbound material specifier(s) per inbound material identifier signifying the material specification. The material demand data may include data on the inbound material amount per inbound material identifier signifying the amount of inbound material to be supplied. The material demand data may specify the production chain(s) of the coating material production 200. The material demand data may include a bill of materials for one or more production chain(s) of the material production 200. The material demand data may include one or more recipe(s) specifying one or more inbound material(s) for production process(es) of the material production 200. The determined material demand data may be provided for access by a supplier system associated with a supplier outside the physical system boundary of the chemical production. Material supply may be triggered by the supplier system accessing the material demand data.

One process step monitored and/or controlled by the operating system 808 may be the feed of inbound materials 9 or the release of produced coating material(s) 10. Another process step monitored and/or controlled may be the collection of coating material data 50. Another process step monitored and/or controlled by the operating system 808 may be the generation of one or more digital access element(s) 40 associated with the coating material(s) 10 produced by the coating material production 200. Another process step monitored and/or controlled by the operating system 808 may be the provisioning of the generated digital access element(s) 40 to data consumer(s), for example via a data providing service under control of the data owner, such as the producer of the coating materials 10 produced by the coating material production 200 controlled by the operating system 808.

Raw materials may include pigment(s), monomers used to prepare polymer(s), solvents and coating material additives. Precursor materials may be intermediate products manufactured from raw material(s). Intermediate products may include polymer solutions and/or polymer dispersions. Intermediate products may include pigment pastes produced from polymer solutions and/or polymer dispersions and pigment(s). Intermediate products may include base varnishes produced from polymer solutions and/or polymer dispersions. The inbound materials 9 may enter the system boundary 1202 of the coating material production 200 at the entry point, such as a resin plant, pigment paste plant, coating material production or a material storage. The inbound materials 9 may be used in the coating material production 200 to produce coating material(s) 10 from the inbound materials 9. The coating material(s) 10 may be produced from inbound materials 9 and/or from intermediate product(s) produced from inbound material(s) or other intermediate product(s). The coating material(s) 10 may be provided on exit points of the coating material production 200. Coating material data 50 may be collected before, during and/or after production of the coating material(s) 10 by the coating material production 200.

Upon producing the coating material 10 or upon exiting of the coating material 10 of the coating material production 200, the digital access element(s) 40 associated with produced coating material(s) 10 may be generated. The digital access element(s) 40 may be generated by an apparatus for generating digital access element(s) 100. The apparatus 100 may be configured to generate the digital access element(s) 40. The apparatus 100 may be configured to receive a request to provide the decentral identifier 20. The apparatus 100 may be configured to generate—in response to the received request—the digital access element 40. A requestor 1206 may be configured generate the request for the digital identifier. Said request may be triggered by a detector and labelling system such as a QR Code reader and generator. For instance. the produced coating material(s) 10 may comprise a physical identifier, such as a QR code, bar code, an embossed code, an optical holographic code, such as zero-order diffractive microstructures, or RFID tag. The physical identifier may be detected, for example by the detector. The detected data may include the batch number and/or a coating material ID. The request to provide the digital identifier may be provided to a digital ID generator 1208 configured to generate the digital identifier 40. The digital ID generator 1208 may provide the generated digital identifier 20 to a digital ID provider 1210. While the digital ID generator 1208 and the digital ID provider 1210 are shown in FIG. 12 as separate units, their functions may be combined within a single unit such that the apparatus 100 comprises a digital ID providing unit configured to generate the digital ID and to provide the generated digital ID.

The digital ID provider 1210 may provide the digital identifier 40 to the requestor 1206. The requestor 1206 may be configured to associate the received digital identifier with the produced coating material 10. The requestor 1206 may hence contain an ID assignor configured to assign the digital identifier 20 to a physical identifier. Such association may include encoding the digital identifier 20 into a code and providing the code for labelling the coating material 10. This way a physical identifier may be provided that relates the physical entity of the coating material 10 with the provided digital identifier 40.

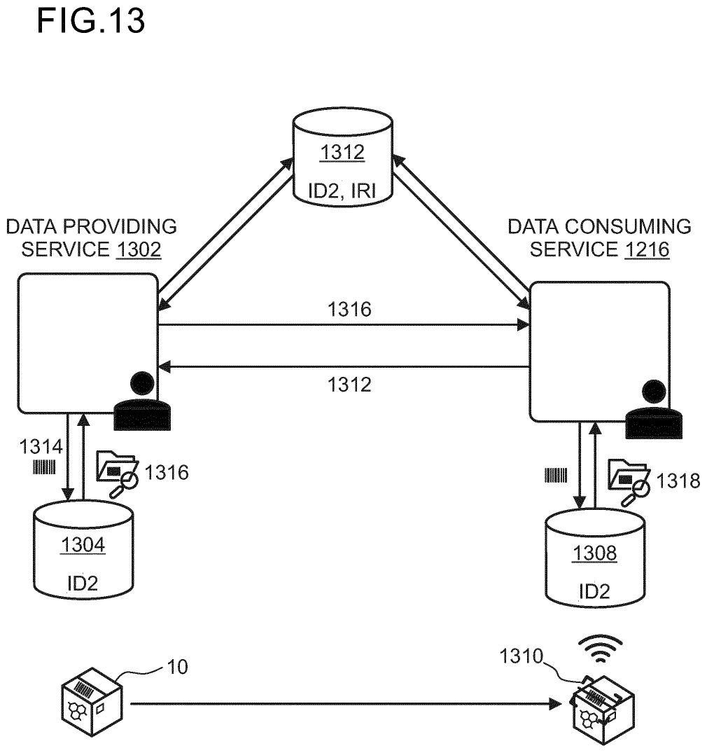

The digital ID provider 1210 may provide the digital identifier 20 to a digital access element generator 1212 configured to generate the digital access element 40 based on the digital identifier 20 received from digital ID provider 310 and access data. The access element generator 1212 may generate the digital access element 40 as described for example in the context of FIG. 1. The generated digital access element 40 may include the digital identifier 20 and access data. Access data may include one or more digital representation(s) pointing to coating material data 50 or parts thereof. The digital access element 40 may include or be related to one or more authentication mechanisms associated with the digital identifier 20 and/or the access data. The authentication mechanisms may be used as described for example in the context of FIG. 13. The digital access element 40 may relate to one or more authorization mechanisms associated with the digital identifier 20 and/or the access data. The authorization mechanisms may be used as described for example in the context of FIG. 13.

The generated digital access element 40 may be provided to a digital access element provider 1214. The digital access element provider 1214 may be configured to provide the digital access element 40 for access by a data consuming service 1216. The digital access element provider 1214 may control the access by the data consuming service 1216. The digital access element provider 1214 may be a data providing service associated with the coating material production 200. The digital access element provider 1214 may be associated with or under control of a data owner of the coating material data 50 associated with the generated digital access element 40. The digital access element 40 may be used to access coating material data 50 as for example described in the context of FIG. 13. FIG. 13 shows a schematic illustration of providing access, via a data providing service associated with a data owner, to a digital access element 40 associated with a coating material 10 using a data consuming service associated with a data user.

The coating material(s) 10 as produced by a coating material production 200, such as coating material production 200 described in relation to FIGS. 2 and 12, may be provided in association with the generated digital access element 40. The digital access element 40 associated with the coating material 10 may be generated as described in FIG. 12. The digital access element 40 may include a digital identifier ID2, such as the digital identifier described in relation to FIG. 12, and access data 30.

The digital access element 40 may further include or relate to authentication and/or authorization information linked to the digital identifier, optionally in terms of the access data 30. The authentication and/or authorization information may be provided for authentication and/or authorization of a data providing service 1302 and/or data consuming service 908. The data providing service 1302 may comprise computer-executable instructions for providing and/or processing data, such as coating material data 50, associated with the data owner, for accessing and/or processing by the data consuming service 1216.

The data owner in this example may be the coating material producer. The data owner may comprise any entity generating data. The data generating node may be coupled to the data owner or the entity producing coating materials 10 from or for which data 50 is generated. The data may be generated by a third-party entity on behalf of the entity producing coating material(s) from or for which data is generated.

The digital identifier ID2 may be connected to coating material data 50 associated with the coating material 10. The digital identifier ID2 may be a Universally Unique Identifier(s) (UUID) or a Digital Identifier(s) (DID). The digital identifier may include any unique identifier uniquely associated with a data owner and/or the coating material. The data owner may be the producer of the coating material(s) 10. Via the digital identifier ID2 and its unique association with the data owner and/or the coating material(s) 10, access to the coating material data 50 may be controlled by the data owner.

The digital access element 40 may be stored in a digital access element storage 1312. The digital access element storage 1312 may be a central repository. The digital access element storage 1312 may be a decentral database. The coating material data 50 associated with the digital identifier ID2 may be stored in a data base 904 associated with the data owner, such as the producer of the coating material(s) 10. The data base may be owned or controlled by the data owner.

The coating material(s) 10 may be physically delivered to a consumer of the coating material(s). The coating material may be connected to a QR-code or comprising an embossed code or an optical holographic code, such as zero-order diffractive microstructures, having encoded the digital identifier ID2. The consumer of the coating material may read the code of the received coating material(s) 10 through a code reader 1310, such as a QR-code reader. The digital identifier may be provided to a data base 1308 associated with the consumer of the coating material(s) 10. The data base may be owned or controlled by the consumer of the coating material(s). The digital identifier may be used to retrieve the digital access element 40 stored in digital access element storage 1312.

In other embodiments the consumer of the coating material may retrieve the digital access element 40 through the decentral data base 1312 based on the data received from the code reader 1310.

Based on the received digital identifier ID2 and access data, a request to access the coating material data 50 associated with the digital identifier ID2 may be triggered by the data consuming service 1216 as signified by arrow 1312. The digital identifier ID2 may be provided to the data providing service 902 associated with or of the producer of the coating material. In addition, authentication and/or authorization information may be provided. Such authentication may be based on a decentral identity of the data providing service 1302 and the data consuming service 1216 and the data related to the authentication mechanism or scheme. If the authentication fails, access to the coating material data 50 may be denied by the data providing service 1302. If the authentication is valid, an authorization step may follow. Such authorization may be based on the decentral identity and the data related to authorization rules. If the authorization fails, access to the coating material data 50 may be denied or access may be adapted. In particular, the authorization as requested may be adapted to be in line with the applicable authorization rules. If the authorization is valid, access to the coating material data 50 may be granted according to the authorization rules as requested.

For access, the digital identifier ID2 may be provided to the data providing service 1302 as signified by arrow 1312. The data providing service 1302 may use the received digital identifier ID2 to retrieve the coating material data 50 associated with the received digital identifier ID2 as signified by arrows 1314 and 1316. The retrieved coating material data 50 associated with received digital identifier ID2 may be provided to the data consuming service 1216 as signified by arrow 1318. The coating material data 50 associated with the received coating material 10 may be stored in the data base 1308 associated with the consumer of the coating material as signified by arrow 1320.

Through the digital identifier ID2, which, as indicated above, denotes a digital identifier 20 as previously described with respect to FIGS. 1 to 10, the coating material data 50 can be uniquely associated with the coating material. Through the decentral network the coating material data 50 may be transferred between the producer of the coating material 10 and the consumer of the coating material 10. This way the coating material data 50 can be shared with unique association to the coating material 10 and without central intermediary directly between the value chain players. This allows to share coating material data 50 in a secure and simple way within the chemical value chain.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

1. An apparatus for generating a digital access element associated with a coating material, wherein the apparatus comprises:
a digital identifier providing unit configured to provide a digital identifier associated with the coating material and coating material data, and
a digital access element generator configured to generate the digital access element based on the digital identifier and access data.

2. The apparatus of claim 1, further comprising a physical identifier providing unit configured to provide a physical identifier for the coating material, wherein the digital identifier is associated with the physical identifier.

3. The apparatus of claim 1, further comprising a collector configured to collect coating material data, wherein the digital identifier is associated with the collected coating material data.

4. The apparatus of claim 1, wherein the digital identifier providing unit is configured to provide the digital identifier upon receiving a request to provide at least the digital identifier.

5. The apparatus of claim 1, wherein the digital access element includes the digital identifier and the access data.

6. The apparatus of claim 1, wherein the access data allow for an access to at least part of the coating material data.

7. The apparatus of claim 1, wherein the digital identifier providing unit is configured to provide the digital identifier based on one or more precursor digital identifiers associated with one or more precursor materials from which the coating material is manufactured.

8. The apparatus of claim 7, wherein the digital access element generator is configured to generate the digital access element based on a combined digital identifier, wherein the combined digital identifier is determined based on the digital identifier associated with the coating material and the one or more precursor digital identifiers.

9. The apparatus of claim 1, wherein the coating material data include data on any of the one or more precursor materials, the coating material itself, a transport of the one or more precursor materials to a manufacturing site of the coating material, or a further transport of the manufactured coating material.

10. An apparatus for manufacturing a coating material, comprising the apparatus of claim 1, wherein the coating material data refer to the manufactured coating material, and wherein the digital identifier is provided based on a physical identifier of the manufactured coating material.

11. An apparatus for managing digital access elements for coating materials generated using the apparatus of claim 1, wherein the apparatus for managing the digital access elements comprises:
a retrieval data storage for storing retrieval data associated with the digital access elements, wherein the retrieval data allow for a retrieval of stored digital access elements and/or for generating digital access elements.

12. The apparatus of claim 11, further comprising:
a digital access elements storage for storing the digital access elements, wherein the retrieval data allow for a retrieval of stored digital access elements from the digital access elements storage.

13. The apparatus of claim 11, wherein the retrieval data comprise digital identifiers and associated access data of coating materials.

14. A method for generating a digital access element associated with a coating material, wherein the method comprises:
providing a digital identifier associated with the coating material and coating material data, and
generating the digital access element based on the digital identifier and access data.

15. A computer program for generating a digital access element associated with a coating material, wherein the program comprises instructions which, when the program is executed by one or more computers, cause the one or more computers to execute the method of claim 14.
